(19) United States
(12) Patent Application Publication
(10) Pub. No.: US 2024/0293073 A1
(43) Pub. Date: Sep. 5, 2024

(54) USE OF ARTIFICIAL INTELLIGENCE PLATFORM TO DIAGNOSE FACIAL WRINKLES

(71) Applicant: Appiell Inc., Plano, TX (US)

(72) Inventors: Sunil Dhawan, Plano, TX (US); Brian Bahram Mahbod, Plano, TX (US); Fauad Hasan, Plano, TX (US)

(21) Appl. No.: 18/573,134

(22) PCT Filed: Jun. 27, 2022
(86) PCT No.: PCT/US2022/035180

§ 371 Date: Dec. 21, 2023

Related U.S. Application Data
(60) Provisional application No. 63/215,161, filed on Jun. 25, 2021.

(51) Int. Cl. A61B 5/00; G06T 7/00; G16H 50/20

(52) U.S. Cl. CPC A61B 5/442; A61B 5/0077; A61B 5/7267; G06T 7/0014; G16H 50/20; G06T 2207/20081; G06T 2207/30088; G06T 2207/30201

(57) ABSTRACT

Disclosed herein are compositions and methods for use in conjunction with assessment of facial wrinkles related to tendon repair. Embodiments disclosed herein can assess facial wrinkles using artificial intelligence platform.

### CROSS-REFERENCE TO RELATED APPLICATION

This application claims priority from U.S. Provisional App. No. 63/215,161 filed on Jun. 25, 2021, the entirety of which is incorporated herein by reference.

### FIELD

The present disclosure generally relates to medical diagnosis technology, and more specifically to the use of artificial intelligence for automated diagnosis of facial wrinkles.

### BACKGROUND

While aesthetic medicine continues to evolve, from non-invasive procedures to surgical treatments, some medical processes remain largely subjective.

For example, physicians typically rely on their experience to diagnose skin issues, which is subjective, inconsistent and carries a relatively high risk of misdiagnosis. Similarly, the medical product industry relies on clinical trials to test new products. The scales used during these trials are also typically subjectively interpreted by investigators and consequently there is a risk of inconsistencies during product development. This not only has a knock-on effect on development timelines but also increases industry costs.

In addition, many patients are unable to self-monitor their skin conditions due to a lack of availability of reliable objective tools. Instead, patients typically rely on consultative sessions with their doctors for all skin assessment needs, which are generally expensive and/or time consuming.

### SUMMARY

Example system, methods, and apparatus are disclosed herein include a computer-implemented facial wrinkle diagnosis platform that employs artificial intelligence to generate objective facial wrinkle diagnosis results efficiently and reliably.

In an example, a method for facial wrinkle diagnosis is disclosed. The method comprises providing, at a user device, instructions indicative of one or more facial poses to be performed by a user. The method also comprises obtaining, via a camera, one or more images of the user performing the one or more facial poses according to the instructions. The method also comprises based on previously collected images of other users performing the one or more facial poses and further based on previously stored user data indicative of facial wrinkle characteristics of the other users, evaluating facial wrinkle characteristics of the user. The method also comprises providing, at the user device, an indication of the facial wrinkle characteristics of the user.

Additional features, advantages, and examples are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not necessarily include all of the advantages and/or aspects listed herein. Thus, the present disclosure expressly contemplates claiming individual and/or various combinations of the aspects and/or advantageous embodiments described herein. Moreover, it should be noted that the language used in the specification has been selected principally for readability and instructional purposes, and not to limit the scope of the inventive subject matter.

### BRIEF DESCRIPTION OF THE DRAWINGS

FIGS. 1, 2A, 2B, 3, 4A, 4B, 4C, 4D, 4E, 5A, 5B, 5C, 6, and 7 illustrate an example graphical user interface of an example mobile application of an example system, in accordance with the present disclosure.

FIG. 8 illustrates an example distributed computer implementation of the example system, in accordance with the present disclosure.

FIG. 9 illustrates example APIs that can be exercised in isolation, in accordance with the present disclosure. FIGS. 10A and 10B illustrate an example dashboard interface of the example system for monitoring and/or generating reports about activity of users of the mobile application, in accordance with the present disclosure.

FIG. 11 illustrates example facial points detected by an example trained machine learning model, in accordance with the present disclosure.

FIG. 12 is a table representative of an example scale for scoring wrinkles associated with a nasolabial fold, in accordance with the present disclosure.

FIG. 13 is a table representative of an example scale for scoring wrinkles associated with crows' feet lines, in accordance with the present disclosure.

FIG. 14 is a table representative of an example scale for scoring wrinkles associated with maximal smiling/muscle contraction, in accordance with the present disclosure.

FIG. 15 is a table representative of an example scale for scoring wrinkles associated with horizontal forehead lines, in accordance with the present disclosure.

FIG. 16 is a table representative of an example scale for scoring wrinkles associated with horizontal forehead lines with maximum brow elevation, in accordance with the present disclosure.

FIG. 17 is table representative of an example scale for scoring wrinkles associated with the nasolabial fold at rest, in accordance with the present disclosure.

FIG. 18 is a table representative of an example scale for scoring wrinkles associated with the nasolabial fold with maximal smiling, in accordance with the present disclosure.

FIG. 19 is a table representative of an example scale for scoring wrinkles associated with marionette lines, in accordance with the present disclosure.

FIGS. 20A, 20B, 2C, 21, 22A, 22B, 22C, 22D, 23, 24A, 24B, 25, 26, 27A, 27B, 28A, 28B, 29, 30, 31, 32, 33, 34, 35, and 36 illustrate another example graphical user interface of an example mobile application of an example system, in accordance with the present disclosure.

FIGS. 37 and 38 illustrate yet another example graphical user interface of an example mobile application of an example system, in accordance with the present disclosure.

FIG. 39 is a diagram of an example facial wrinkle diagnosis system, according to an example embodiment.

FIG. 40 is a flowchart of an example process performed by a facial wrinkle diagnosis system, according to an example embodiment.

### DETAILED DESCRIPTION

Some examples herein include a system and associated application that allows a user to take facial photographs using a mobile phone, and in real-time get facial wrinkle severity assessment computed using an artificial intelligence platform which has been trained by top experts in aesthetic dermatology, without necessarily requiring the user to make an appointment with an aesthetics practitioner to assess his/her face. Thus, some examples herein may involve providing facial wrinkle diagnosis and/or assessments based on an objective and unbiased wrinkle classification method.

Some examples herein include a unique deep-learning system that objectively assesses and grades facial lines, wrinkles and other skin problems. In one example, a machine-learning based system is provided that is trained by some of the leading dermatologists in the world, and can be used for multiple use cases across the aesthetics medicine industry. For example, physicians may benefit from the example systems and methods of the present disclosure. For instance, some example systems disclosed herein collate the collective, diagnostic experience of world renowned doctors, in objectively assessing skin disorders. Our deep learning platform also provides adaptive pedagogical functionalities, which will serve as an invaluable training tool for practicing physicians. Skin care companies and product developers may also gain from our expertly trained neural network technologies. Example machine learning algorithms disclosed herein may ensure that researchers have access to higher quality, compartmentalized and timely data—bringing precision to development and helping reduce costs. Examples in the present disclosure may also facilitate providing time-saving participant screening capabilities—thus reducing product development time and trial costs. Some examples disclosed herein include an interactive platform that allows for a more robust physician/patient relationship through expanded access to care—patients are able to self-monitor their skin conditions using example mobile platforms disclosed herein, while physicians are automatically kept updated on patient progress. This enhanced connection improves both clinical workflows as well as eliminates unnecessary visits to the doctor—these visits can be scheduled only when treatments are recommended by the system.

“Machine learning” or “ML” includes the use of computational and statistical tools, or algorithms, for identifying relationships in data and making intelligent predictions. Within the umbrella term of machine learning, deep neural networks are a specific type of machine learning that have been applied to image recognition. A wide variety of other models of machine learning exist and are chosen as appropriate for the particular task and characteristics of the data.

The term “Fully supervised learning (FSL)” may include an approach where teaching the model involves using data where each datapoint is labeled with an output. For example, clinically annotated or diagnoses associated with images of facial wrinkles. Unsupervised learning or ‘Self-supervised Learning (SSL)” occurs when the model trains on the data itself, without any labeling.

### Platform

Some examples disclosed herein involve training a deep learning engine to generate a unique protocol for classification.

Some examples herein involve generating a developer-neutral and multi-racial set of scoring scales. For example, some examples herein involve generating a set of scales for scoring glabellar lines/rhytides. The generated scales can then be used for training and validating the artificial intelligence engine that can then be used for scoring new photos by assigning a score based on this validated scale.

Multiple companies have developed their own glabellar line scale, but these scales are proprietary and others generally cannot use them. Furthermore, each physician has to learn each scale, which is a major burden, as some have a 4 point scale, some a 5 point scale, etc. Another issue is that generally these scales are only based on Caucasian population, predominantly female. Given the multi-ethnic population of the United States, as well a larger number of men being treated for their glabellar lines, we will use photos from multiple skin types, and both men and women.

### Study Protocol

Recruit a diverse population and take pictures in repose and active (or appropriate poses for each area). Subjects may sign a consent form prior to photos being taken.

Photos are taken at full frontal view, including the upper half of the face, including a line that goes from medial canthus to medial canthus, to the upper forehead, to include the entire glabellar complex, and all lines/rhytides. Adequate lighting is provided.

### Table Classifying the Skin Types for Use in the Appiell System

Select 5 Key opinion leaders (KOL) who have done at least three clinical trials in the use of grading the glabellar area for multiple toxin trials, have published in the field of glabellar lines, have lectured at national meetings on glabellar lines, are recognized by national societies/professional organizations as being know experts in the glabellar area, etc.

The photos are provided for scoring through an example system to each of the selected experts (KOL). The pictures in which less than 80% of KOLs show consensus are pulled out for a live collective scoring session so consensus can be achieved.

These consensus photos with scales are then used by some example systems herein to generate a Glabellar Scale.

### High Level System Flow

An example mobile application (“front-end”) disclosed herein can be developed using the React Native framework so the application can be developed once and run on both Apple iPhone and various Android based platforms, and have a common look and feel, to the extent possible, regardless of platform. In other examples, different development platforms can be alternatively or additionally used to develop the mobile application. Certain native sub-modules are used as users on a particular platform have familiarity with such sub-modules in other apps on that platform (e.g., calendar date selector).

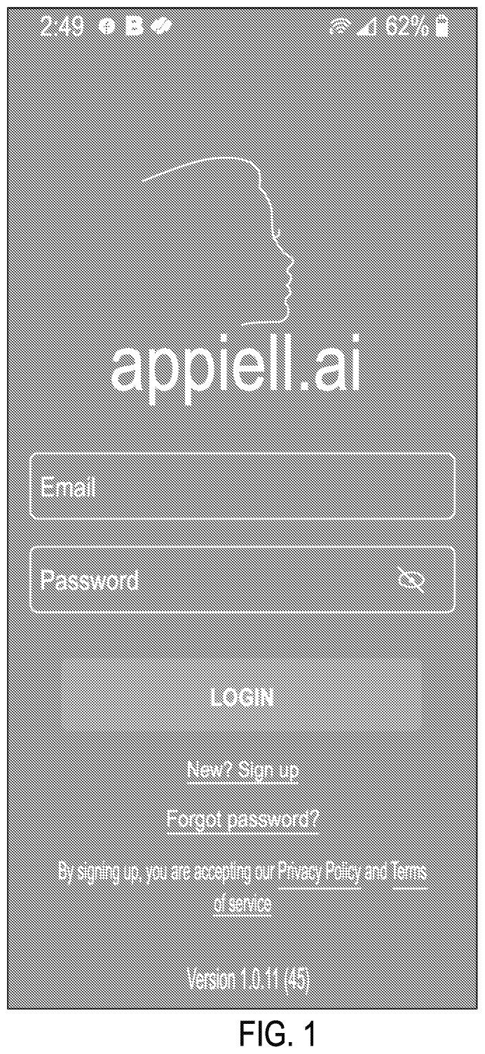

FIGS. 1, 2A, 2B, 3, 4A, 4B, 4C, 4D, 4E, 5A, 5B, 5C, 6, and 7 illustrate an example graphical user interface of the example mobile application. In this illustrated example, information is flowing in an example system that includes one or more servers (not shown) and the example mobile application (e.g., installed on one or more client devices (not shown)) as follows:

As shown in FIG. 1, a user signs up to the example system, and afterwards can sign into the system.

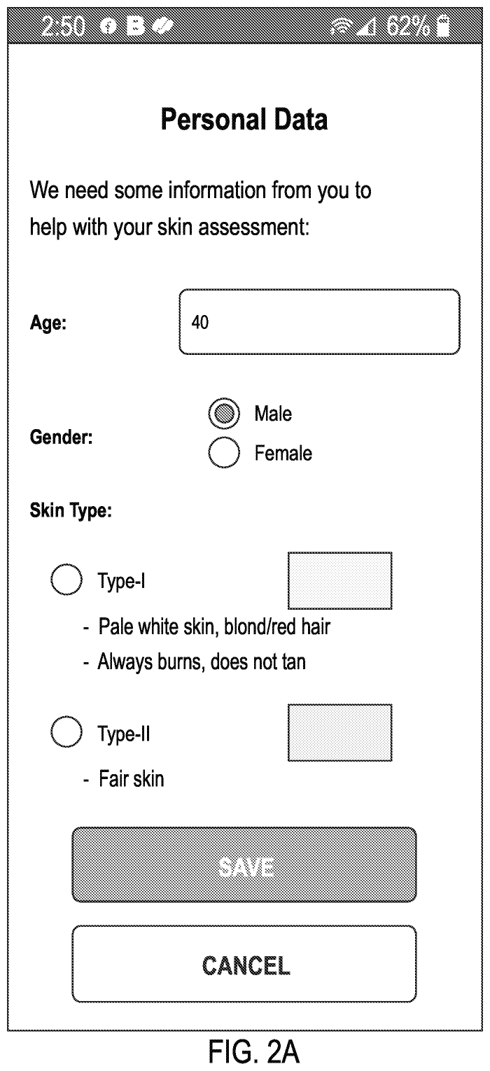

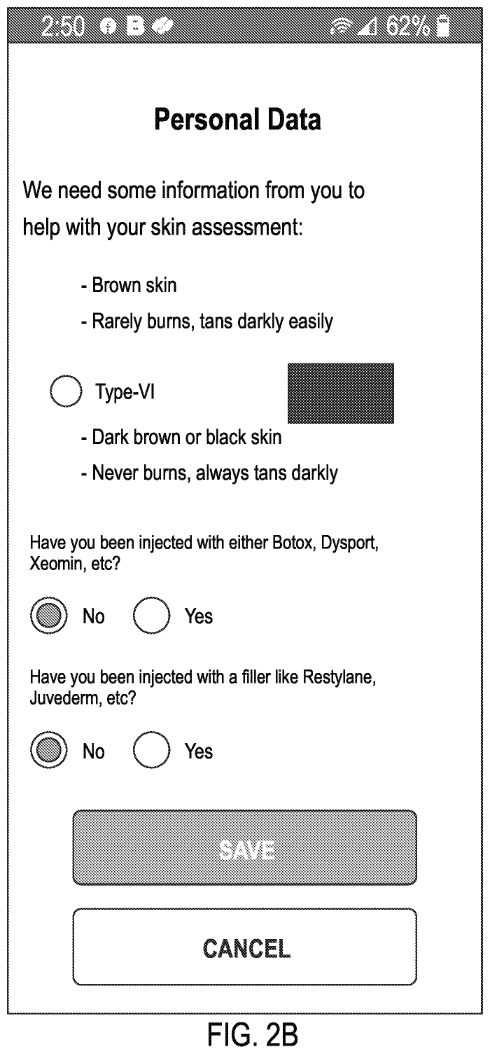

Upon the first sign in, the user is presented with a profile page to fill out (FIGS. 2A and 2B). The user is asked to fill out his/her age, gender, choose her skin type by using the skin color that matches with the closest color out of the six colors presented. Note that the user can come back to the profile tab and change her settings at any time. Further, the user is asked about any previous use of “injections” and “fillers” (FIG. 2B). If the user selects “Yes” (in FIG. 2B), then the user is presented with a list to choose from for each, and then a list of the areas the injection and/or filler has been administered. The user is also given the option of entering her own injection, filler, and the corresponding areas if that option was not presented to her via the list.

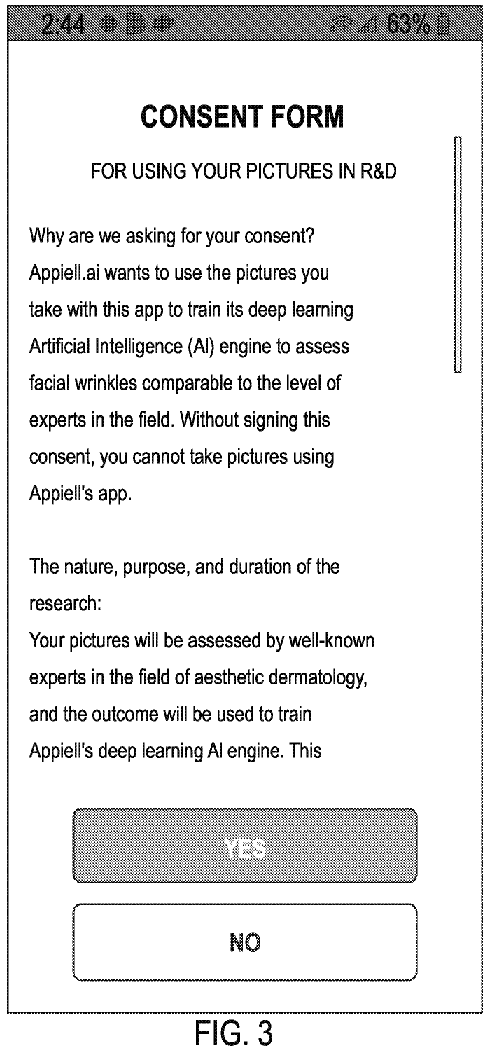

If the user has not previously signed the consent form for use of his/her picture that she takes with the app, the user is presented (FIG. 3) with the consent form to sign. If the user does not sign the consent form, the user cannot proceed.

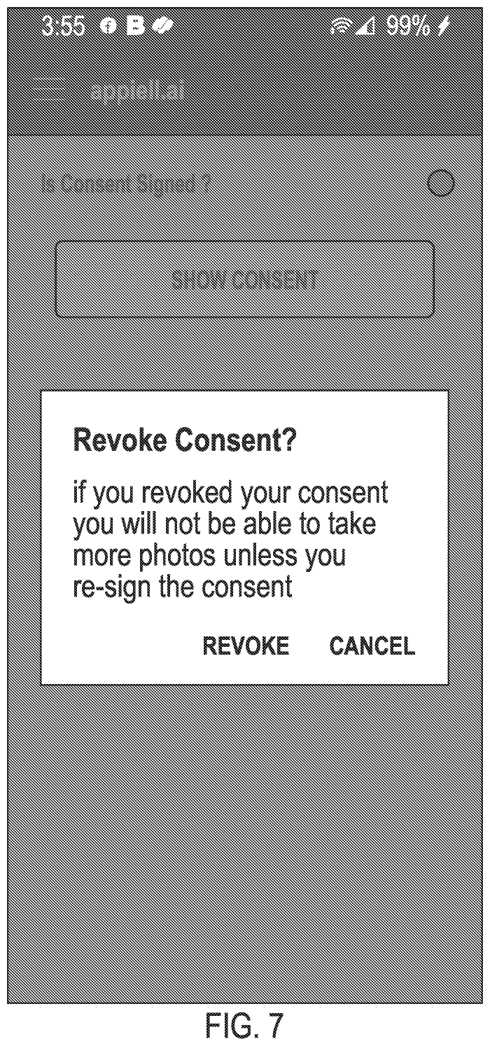

If the user subsequently revokes her consent (FIG. 7), the user's pictures will no longer be used or scored by the example system.

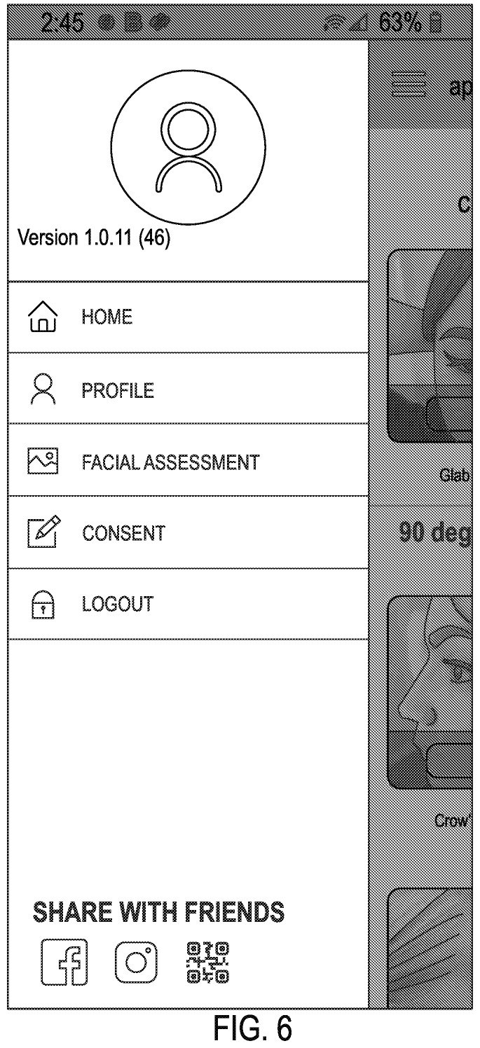

The user is optionally then asked (by the system) to indicate if she is interested in participating in future clinical trials by drug development partners. The user can answer yes or no, and can change that answer in the profile at any time (FIG. 6).

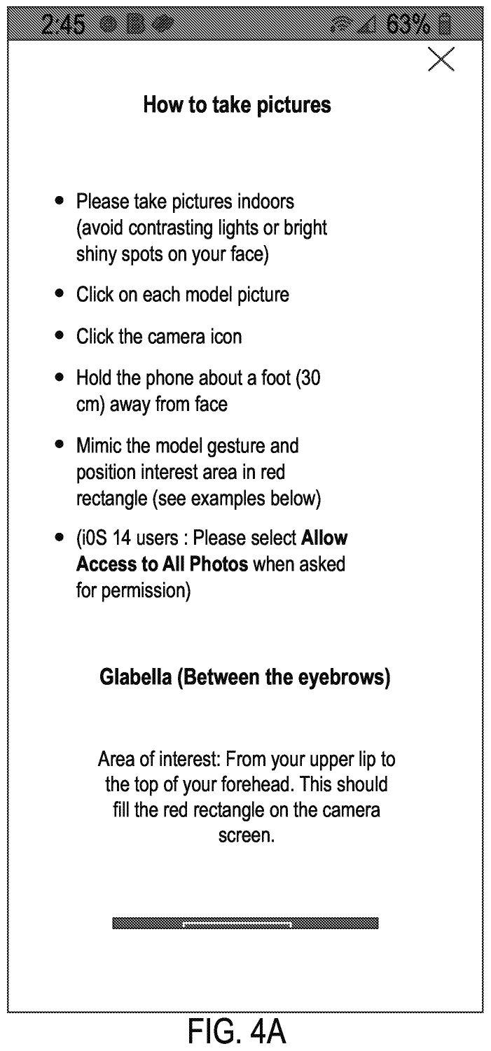

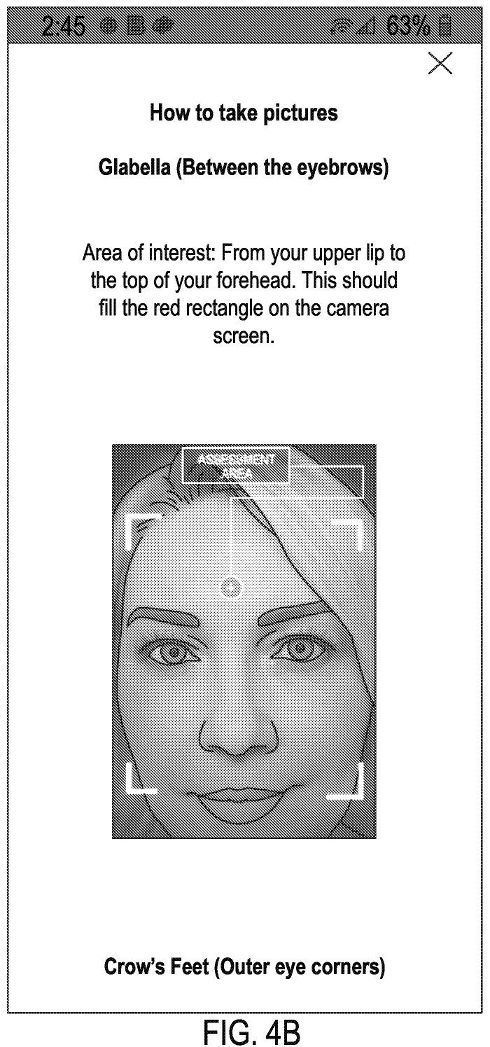

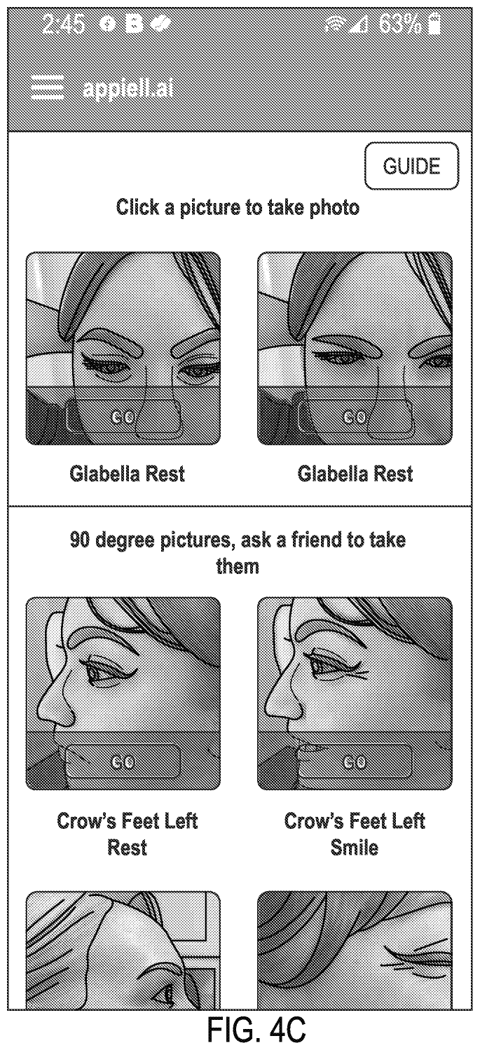

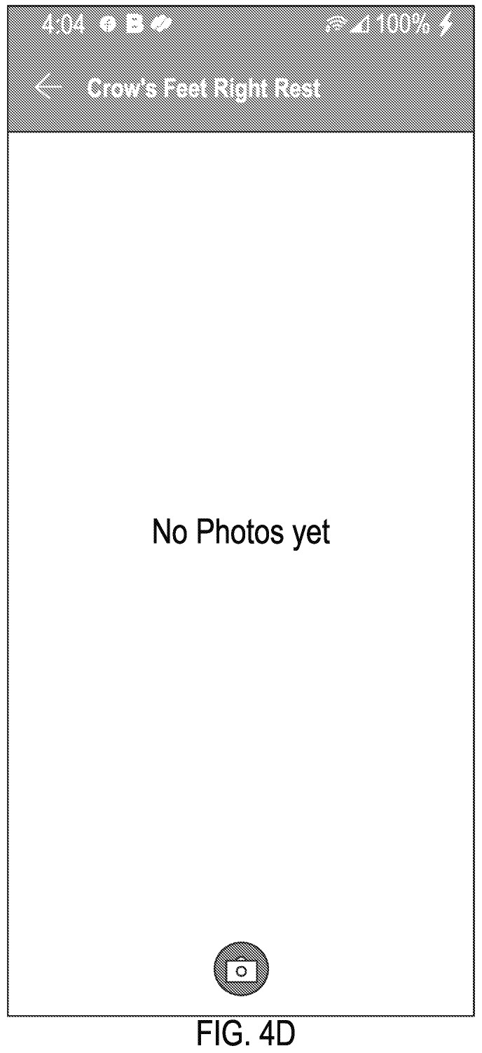

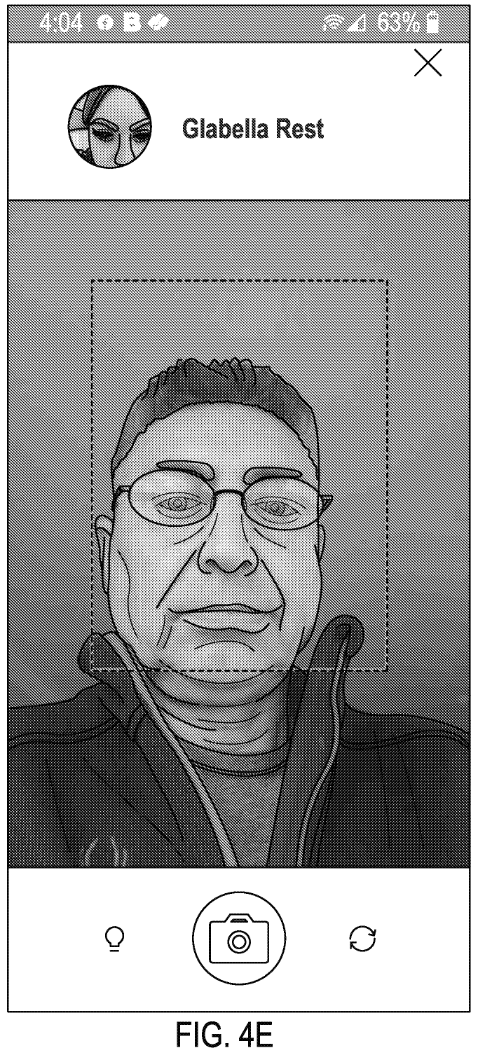

Next, the user is presented with a Guide on how to take pictures that are appropriate for the deep learning engine (FIGS. 4A-4C). The Guide gives clear instructions, with a model's picture (FIG. 4B) showing samples on how to take pictures.

Next, the user is presented with model's picture for the appropriate pose (FIG. 4C), so she can click on that picture, and take that particular pose.

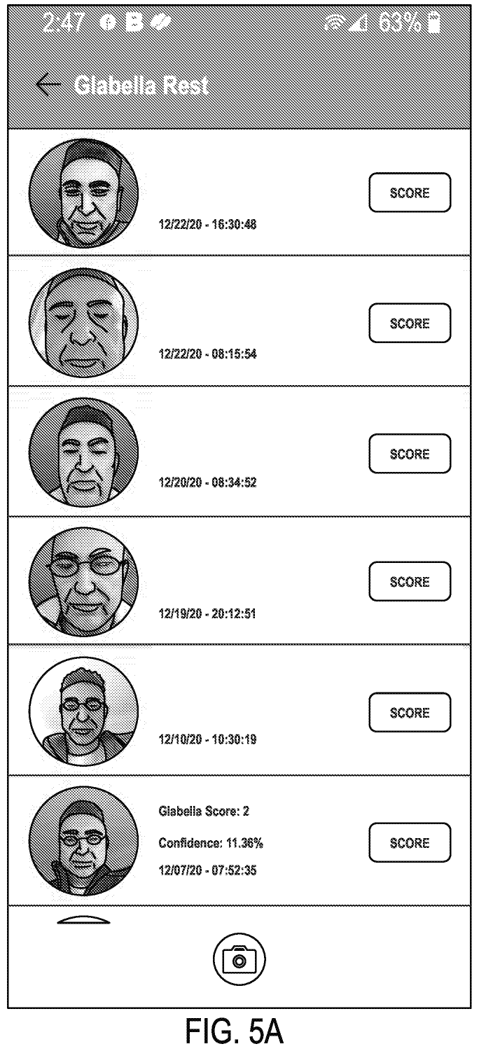

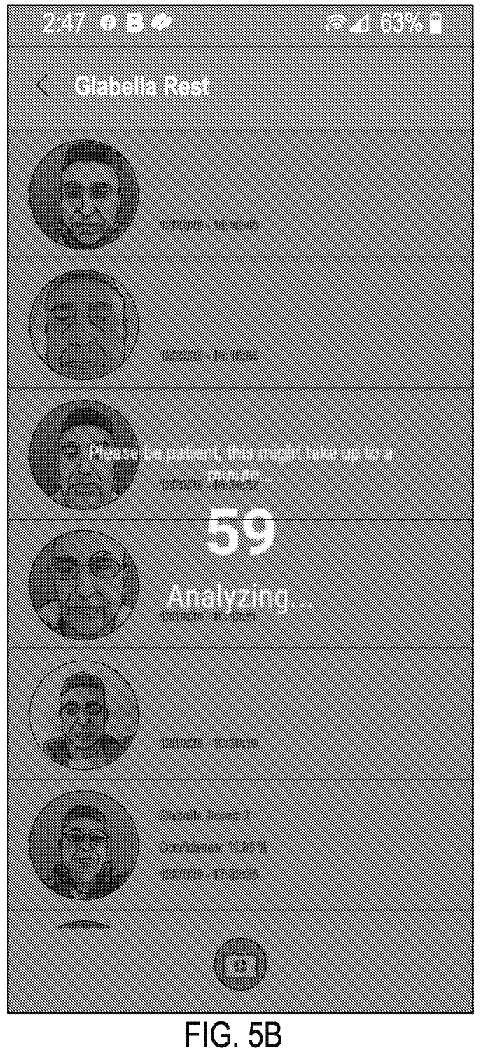

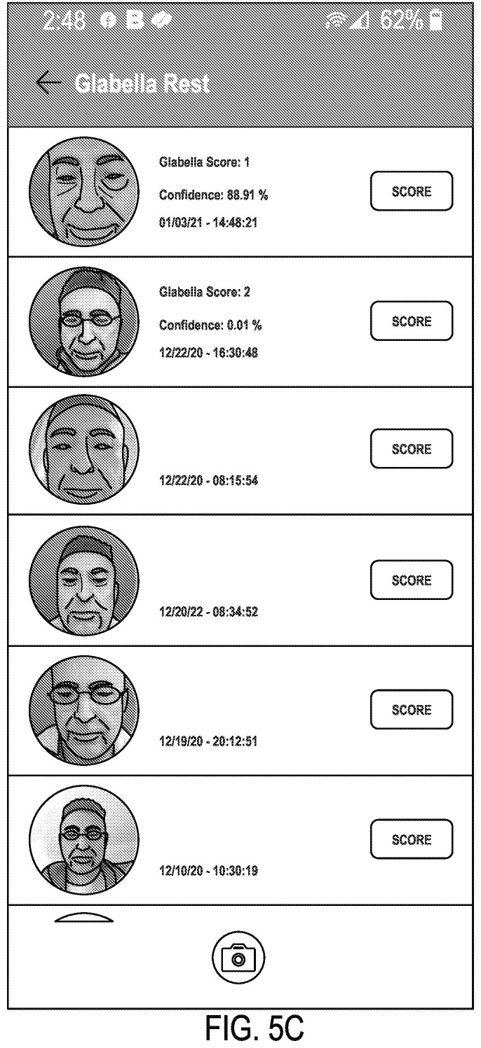

In some examples, once the picture has been taken, a SCORE button appears next to the picture (FIG. 5A). (this could have other variants in other examples, but conceptually it is the same). This SCORE button is intended for the user to self-evaluate using the AI/Deep Learning engine to receive a score of 0 (no wrinkles) to 3 (maximum wrinkles). This process will take several seconds. The user is presented with a countdown counter (FIG. 5B) so the user knows activity is happening on the server side.

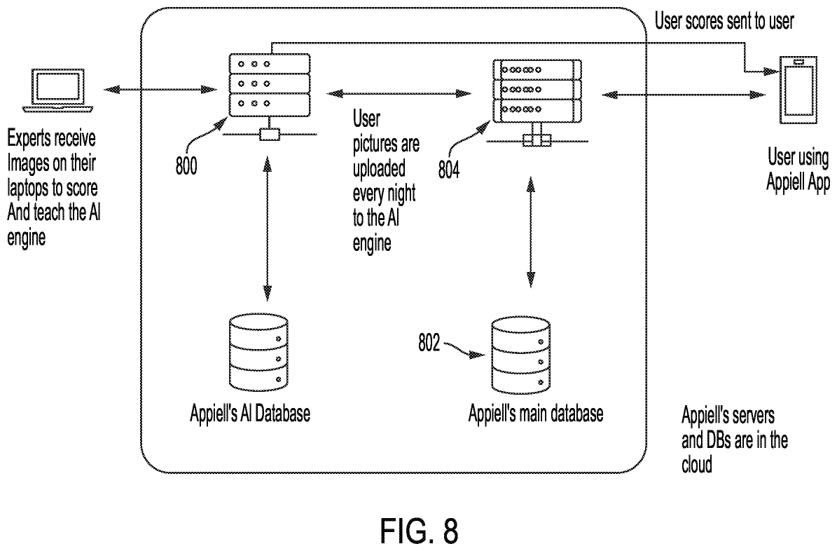

FIG. 8 illustrates an example distributed computer implementation of the example system, in accordance with the present disclosure.

The taken picture is transmitted to an example server (FIG. 8) and and/or one or more example databases 802 (e.g., “main database” shown in FIG. 8), along with attributes of the user (age, gender, skin type, and type of picture—e.g., glabellar frowning).

Every night (and/or intermittently, periodically, according to any other schedule, etc.), a background server 802 extracts the pictures collected during the last 24 hours, and uploads the pictures to an example deep learning/AI engine 804 for analysis (FIG. 8).

In the learning phase, each picture is presented in a separate “scoring system” to each expert aesthetics physician for scoring.

As each physician scores each picture, in some examples, when there is 80% consensus, the engine learns that the given score is the correct score. The other 20% opinions are discarded in favor of the 80% opinion.

In some examples, if there is no 80% consensus, then that picture is isolated, and along with other pictures with less than 80% consensus, they are presented in a live session to the collective body of the experts so consensus can be achieved, after which the engine is trained by it.

### The Server Side (Back-End) Details

In some examples, the system can be implemented using the Python language along with the Django framework. Use of Django may facilitate creation of structured database schema without the need for the database schema to be created separately. In other examples, different database and/or programming language frameworks can alternatively be used to implement the example system (and/or portions thereof). Upon creation of the schema, the example system also captures the data dependencies, allowing easier modification to data and schema when needed. In some examples, the system is implemented using the open source Postgres relational database for data permanence. In other examples, different databases can be alternatively used for implementing the examples system (and/or portions thereof).

In some examples, one or more databases of the example system are hosted in the cloud at a hosted service provider. The hosted database service can then be used, for example, to take regular data backups via the service provider. Similarly, in these examples, the task of restoring to a previous version of the data that had been stored in the database can also be performed (e.g., via the service provider).

### Example Programming Methodology—System Isolation

In some examples, an example server device herein (back-end) is isolated from the mobile application (front-end) using a set of well-defined Application Programming Interfaces (APIs). These APIs are documented within software code of the example system. These APIs isolate the app from the logic in the backend server, so not only can the server side code be improved, but also this allows the example system to offer its service of taking facial pictures and scoring them according to its own protocol to other potential partners who have a large installed base for their app(s).

In addition, with this arrangement, when new software engineers join the development team of the example system, the learning curve of the new software engineers may be reduced substantially, and a new engineer can quickly ramp up his learning process.

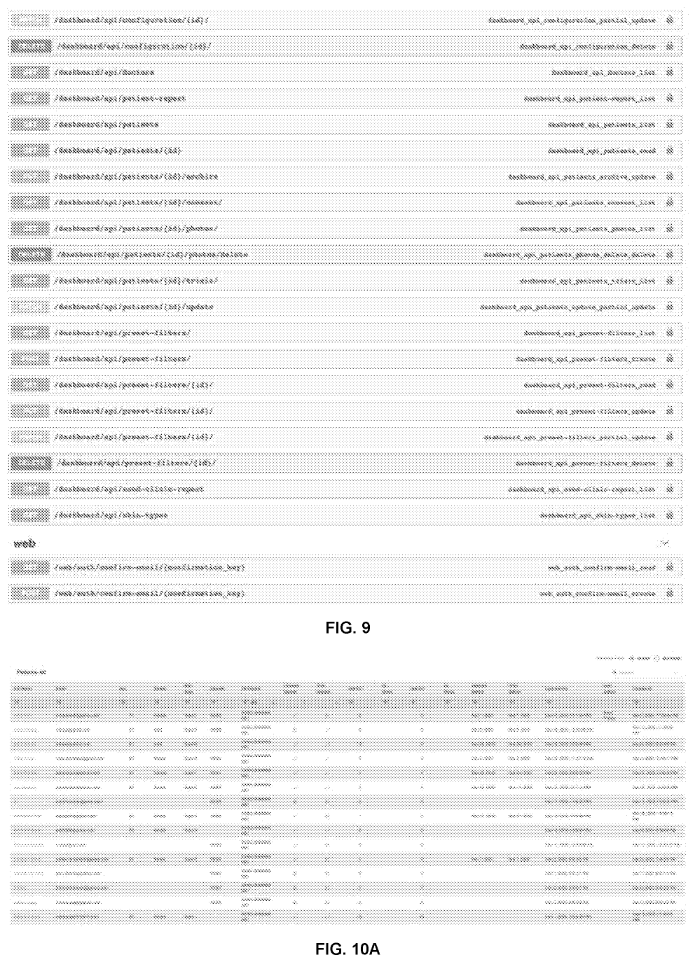

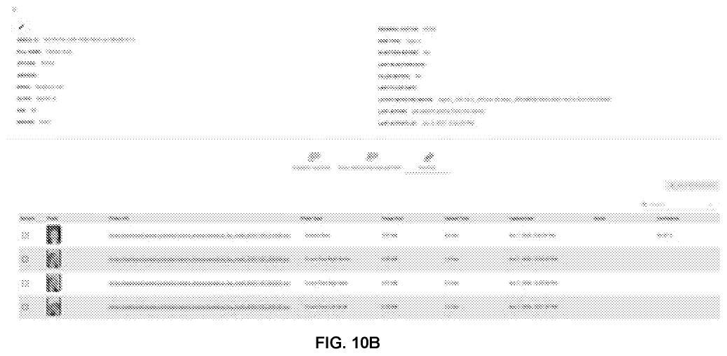

FIG. 9 illustrates example APIs that can be exercised in isolation, in accordance with the present disclosure. Other example APIs are possible alternatively or additionally to the example APIs shown in FIG. 9.

### System Security

The system is designed to be secure. The main database (FIG. 8) that contains all user data is isolated from external access, and may be configured to respond to requests from the servers of the example system with known identifiers. The AI database (FIG. 8) may be configured to store data for analysis and/or other machine learning functions of the present disclosure. Prior to analysis, pictures received from users may be cropped so user identities are not revealed (e.g., only demographic data). Subsequent to training, the pictures may be removed from the AI database, as they are no longer needed. In some examples, pictures and/or other user information may remain stored in the main secure database (FIG. 8) as long as the user's consent is in force. It is noted that the terms “picture,” “photo,” and “image” may be used interchangeably in the present disclosure in reference to one or more images captured by a user of the example system via the example mobile application.

Some example systems and methods herein include a two-factor identification process (via sending a dynamically generated code to the user's mobile device in order to validate the user) should the user opt for 2-factor identification.

Prior to completion of the user sign-up (FIG. 1), in some examples, a user's email is validated by asking the user to click a validation link from within the example mobile application.

### Automated Reporting of User Activity

FIGS. 10A and 10B illustrate an example dashboard interface of the example system for monitoring and/or generating reports about activity of users of the mobile application, in accordance with the present disclosure. In some examples, the system provides the example dashboard (FIGS. 10A and/or 10B) for generating automated reports to the participating practitioners on the activity of their users within the mobile application. In some examples, the report is sent out via email, with a summary, and a CSV and PDF attachment containing the details. Each report may be customized via a report generation dashboard (FIG. 10B), with the recipient, the contents of the report, and the frequency of the report fully customizable. An example of such a report is shown in FIG. 10B.

In some examples, the system additionally or alternatively generates a master report, showing in a table the summary of activity of users related to each participating practitioner.

### The Back-End Monitoring Dashboard

A comprehensive example dashboard (FIGS. 10A-10B) system is disclosed herein that allows sorting and combination filtering of the majority of the parameters in the system. In some examples, the dashboard system may be implemented as a web-based system with multiple levels of security privilege. From this dashboard, the operator can drill down to the details of each user, and see a complete status of the user, including the user's profile, the status of the user consent, the pictures taken, the device the user is on, the referring practitioner, if the user joined the system via a practitioner, etc. A screenshot of the example dashboard is shown in FIG. 10A, along with a screenshot of the user detail page (FIG. 10B).

### Social Sharing

In some examples, the mobile application of the example system include an interface for a user to share information via social media, including but not limited to Facebook, Instagram, and QR code sharing, and/or allowing a user to share the example mobile application (e.g., information about the mobile application, a download link or other URL link associated with the mobile application, etc.) via social media with friends, so that a friend of the user can take advantage of the opportunity of assessing her face in real time, by using the example system disclosed herein including the machine-learning engine trained by top experts in aesthetics dermatology.

Some example methods disclosed herein involve administration of a neurotoxin to a muscle, such as, for example, a muscle attached to a tendon. Another example method disclosed herein involves detecting and/or analyzing crow's feet lines.

### Example AI/Deep Learning Methods

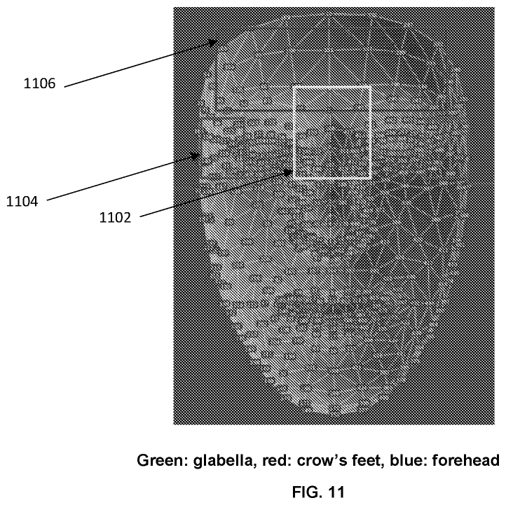

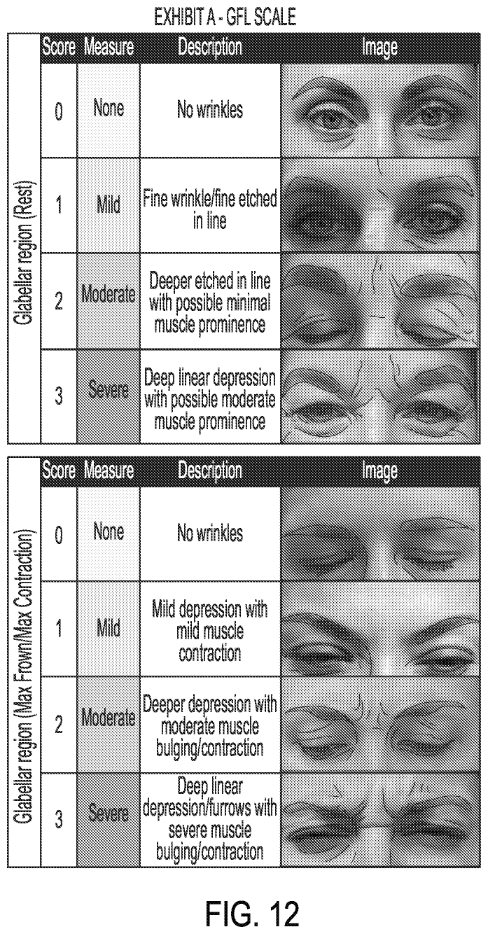

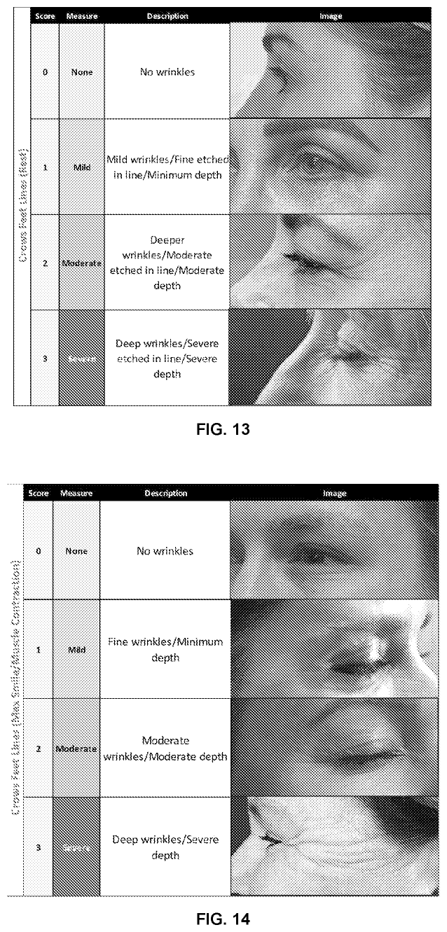

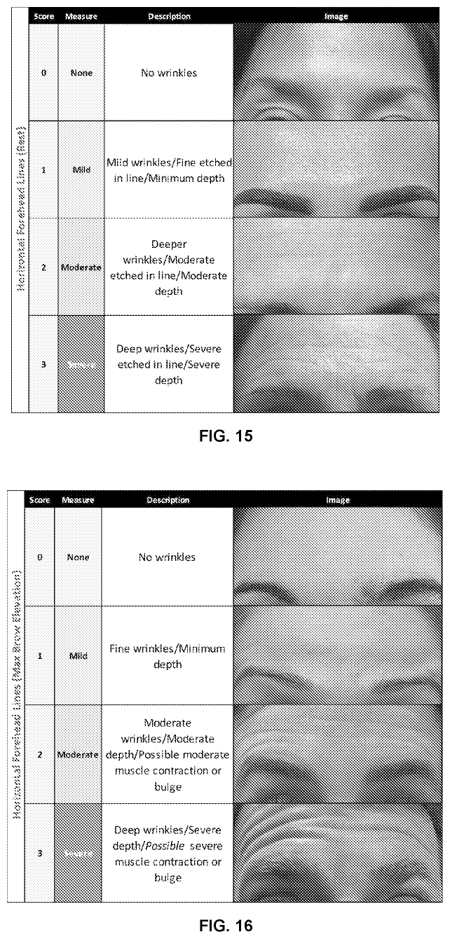

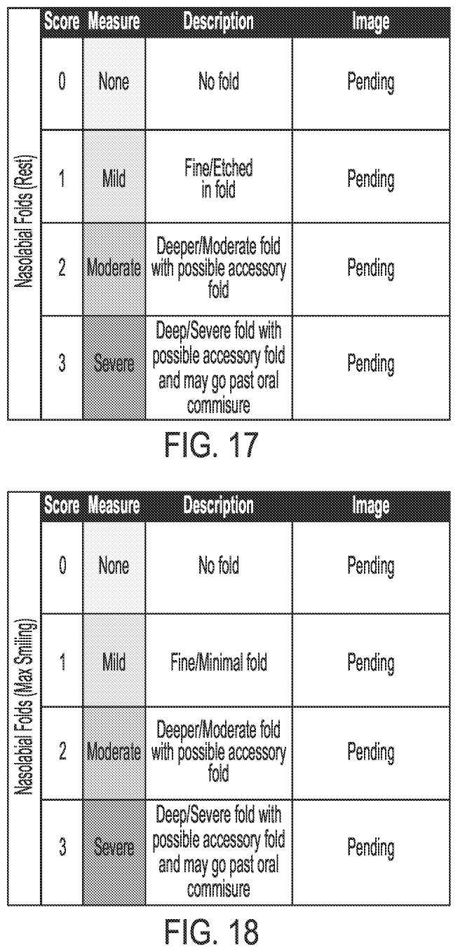

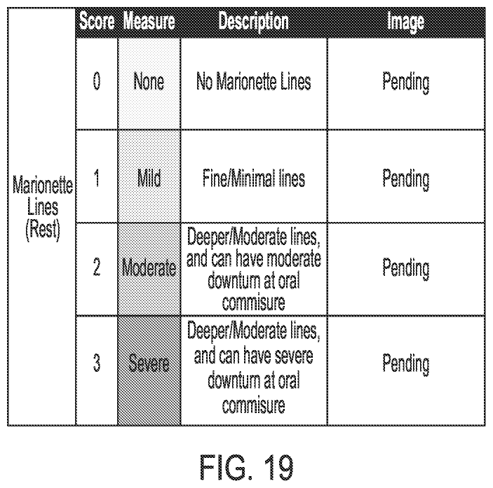

FIG. 11 illustrates example facial points used for training an example machine learning model, in accordance with the present disclosure. FIGS. 12, 13, 14, 15, 16, 17, 18, and 19 illustrate example scales used for classifying and/or scoring various different types of facial wrinkles (e.g., crow's feet lines (CFL), horizontal forehead lines (HFL), nasolabial fold (NLF), marrionette lines (ML), etc.), in accordance with the present disclosure.

Some examples disclosed herein involve image classification using a set of training images associated with multiple classes. For the glabellar lines case, for example, those classes are mapped to a set of discrete scores (e.g., FIGS. 12-19) related to the severity of glabellar lines, namely zero (normal, no wrinkles) to three (severe).

An example method disclosed herein first involves cropping users' images as required by the use case. For example, in the glabellar lines use-case, a forehead image may be cropped or selected from one or more full facial images. Continuing with the example method, a pre-trained facial points detector is then applied to detect facial points in the forehead image (and/or other images). For instance, in the illustrated example of FIG. 11, 468 facial points are detected by the example pre-trained facial points detector. In the example method, a region-of-interest (e.g. glabella 1102, crow's feet 1104, forehead 1106, etc.) is then cropped or selected based on the corresponding coordinates (e.g., the points illustrated in the rectangles in FIG. 11). In some examples, the regions of interest shown in FIG. 11 may be selected manually by an expert as a series of coordinates corresponding to a portion of a face associated with the region of interest. In some examples, the face mesh model can be generated using a modeling algorithm (e.g., MediaPipe FaceMesh). Thus, for example, each rectangular region shown in FIG. 11 may represent a set of coordinates corresponding to a respective region of interest.

In the example method, experts may annotate a set of images by looking at either or both at the full face and/or the forehead, crow's feet, glabella, etc., regions by scoring each image (or cropped image) according to a given range (e.g., the scales of FIGS. 12-19, etc.).

In some examples, the method may include classifying training images into the required classes using a deep Convolutional Neural Network (CNN). In some examples, some of the training images (e.g., annotated images) may be used as a validation set and/or a test set in order to avoid over-fitting issues. In some examples, the method includes transfer learning, namely fine-tuning of a pre-trained model to the given domain (e.g. glabellar lines) to compensate for a lack of millions of annotated images required for a from-scratch end-to-end training of a deep convolutional network. In some examples, the method further includes hyper-parameter optimization to obtain an optimal set of parameters (learning-rate, size of the pre-trained model etc.) and therefore achieve optimal results given a particular number of annotated images.

In one example, a pre-trained facemesh detector (e.g., mediapipe facemesh, etc.) may be run on all the annotated images. In this example, the method may thus generate a set of 468 points (per face) aligned to the actual facial landmarks (e.g., regions of interest, etc.). For instance, an index of the nose's tip may be mapped across all the annotated face images by the number ‘4’ shown in FIG. 11.

In some instances, the method may then involve cropping regions of interest in each image (e.g., glabella, crow's feet, forehead, etc.) based on the sets of coordinates.

Continuing with this example, the method may also involve splitting the annotated (and/or cropped, aligned, etc.) images into a training data set, a validation data set, and a testing data set, by seeding the process in order to get repeatable splits.

### Fine Tuning Process

Thus, the method may involve pre-training a neural network model on a source dataset as noted above, or using a pre-trained dataset (e.g., trained on a huge dataset). For instance, one or more pre-trained convolutional models can be adapted for use in the method (e.g., ResNet, DenseNet, Facenet, etc.) and/or a visual transformer model (ViT) that have been trained on a large dataset, such as the ImageNet dataset or the VGGFace2 dataset for example.

In an example, the method may involve creating a new neural network model (e.g., the target model). The method may then involve copying all layers and corresponding parameters of a pre-trained model (except the output layer). For instance, the pre-trained model parameters may contain knowledge learned from a source dataset which may also be applicable to a target dataset being generated for the method of the present disclosure (e.g., glabellas, forehead, crow's feet, etc.).

The method may then involve adding an output layer to the target mode, whose number of outputs corresponds to the number of categories of the target dataset (e.g., 4 categories corresponding to the 4 grades in each of FIGS. 12-19, etc.). The method may also involve randomly initializing the model parameters of that output layer.

The method may then involve training the target model on the target training dataset (e.g., glabella, forehead, crow's feet dataset, etc.). In some implementations, the method may involve freezing all the neural network layers except the output layer during this training step (e.g., so as to train the output layer from scratch until it is performing “well” without changing the parameters of the other layers, etc.), then unfreezing the other layers and retraining the whole model (optionally with the “unfrozen” layers having a lower learning rate).

In some examples, instead of or in addition to training a single classifier per input region, the method may involve training a multi-task classifier that takes all the regions of interest as input. In that case, the multi-task classifier may benefit from features across the face even those that are outside a specific region of interest (e.g., glabellar lines of grade 3 may be related to forehead lines of type 2, etc.).

### Fully-Supervised and Self-Supervised Training of the Deep Learning Machine

Some example systems disclosed herein use fully-supervised learning (FSL), self-supervised learning (SSL), and/or both FSL and SSL for training of the AI engine (FIG. 8).

In some examples, FSL methodology is used, by selecting a training set of pictures scored by the experts using example scales disclosed herein (e.g., one or more of the scales illustrated in FIGS. 12-19), which may be developed collaboratively by one or more experts. In some examples, additionally or alternatively, SSL is used to let the machine learn on its own.

Example inputs to the AI machine of the example system may include thousands of scored pictures taken in clinical settings that are already scored by the experts. In some examples, these pictures may be presented to the experts via a web interface of the example system, which may provide magnification features and/or other features to enable honing in on particular areas of the pictures displayed to the expert(s). Via the example interface disclosed herein, the experts may score the pictures collaboratively, and agree on the score, for example. In some examples, these pictures are then used for one or both of the FSL and SSL phases of the training process of the AI engine of the example system. In some examples, the AI machine goes through a number of stages (in the FSL) phase to determine the correct algorithms. In some examples, the process described above is applied to train a machine learning model of the present disclosure for detecting and/or classifying wrinkles associated with facial and/or other body areas.

In some examples, the images stored in Appiel's database (FIG. 8) may be additionally or alternatively classified (in the machine learning model) based on skin type, gender, 4-point FWS severity (e.g., grade 0-3), age group, zip code, and/or other demographic information. These parameters may further improve the accuracy of the machine learning model.

In some examples, an example database of the example system of FIG. 8 may also store information such as dosing, product, onset time of product (e.g., how quickly a user got to a lower grade, etc.), duration of effect (e.g., how long did the effect of using the product at a certain grade and at what time did user start moving to each of the higher/lower grades after using the product, etc.), naïve or past treatments by the user, and/or pricing information. In some instances, this information can be collected from the users and/or used to train the machine learning model to predict similar outcomes and/or optimal methods of treatment for other users having similar conditions (e.g., age groups, wrinkles, etc.).

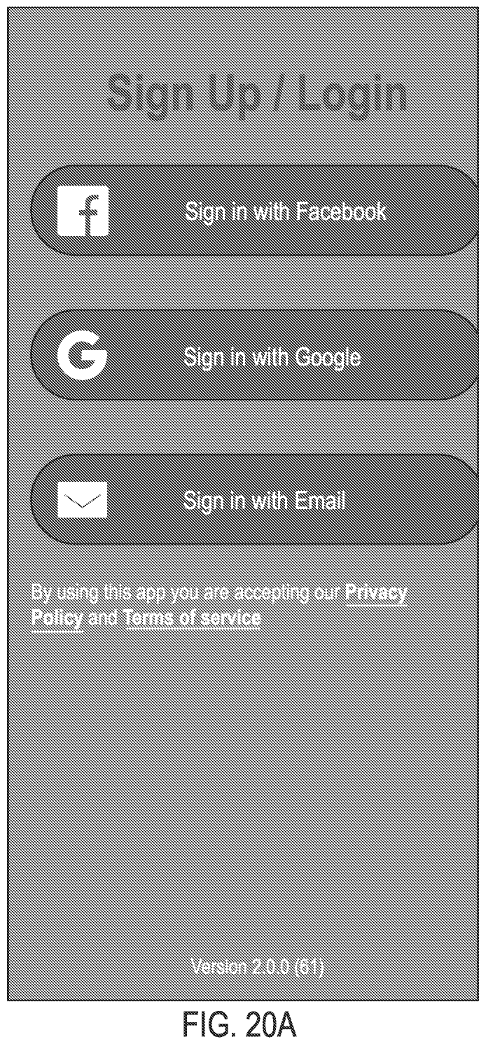

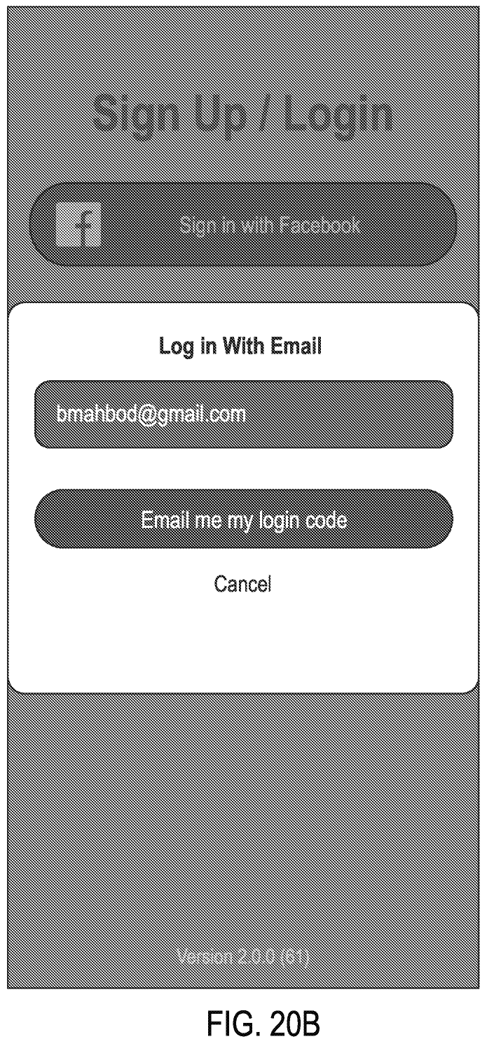

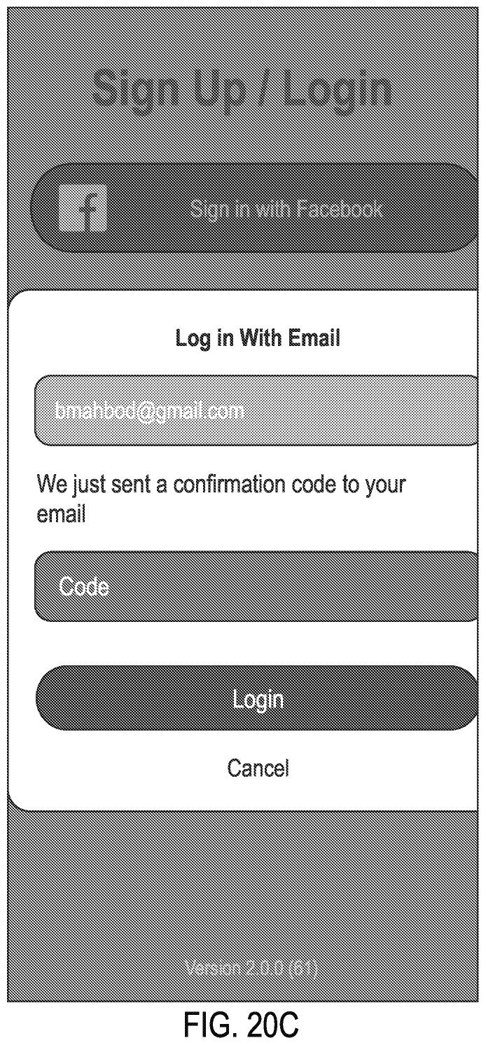

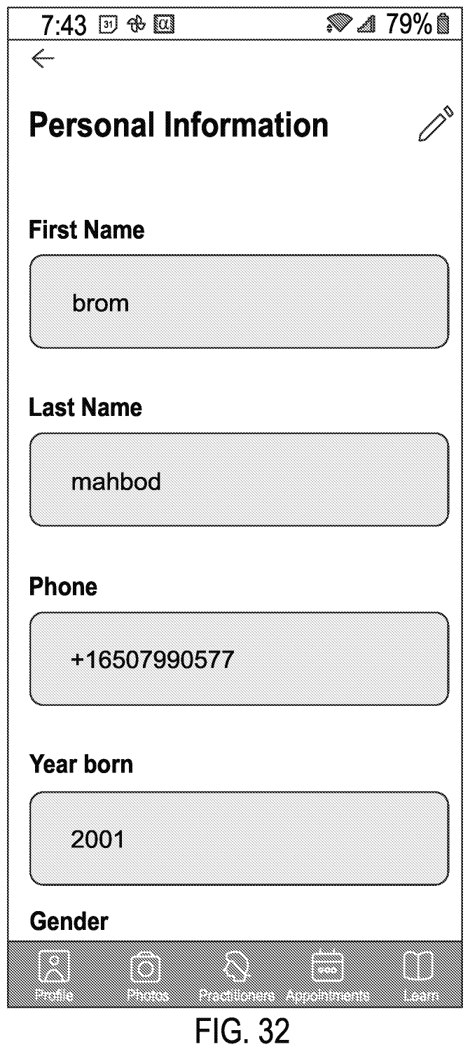

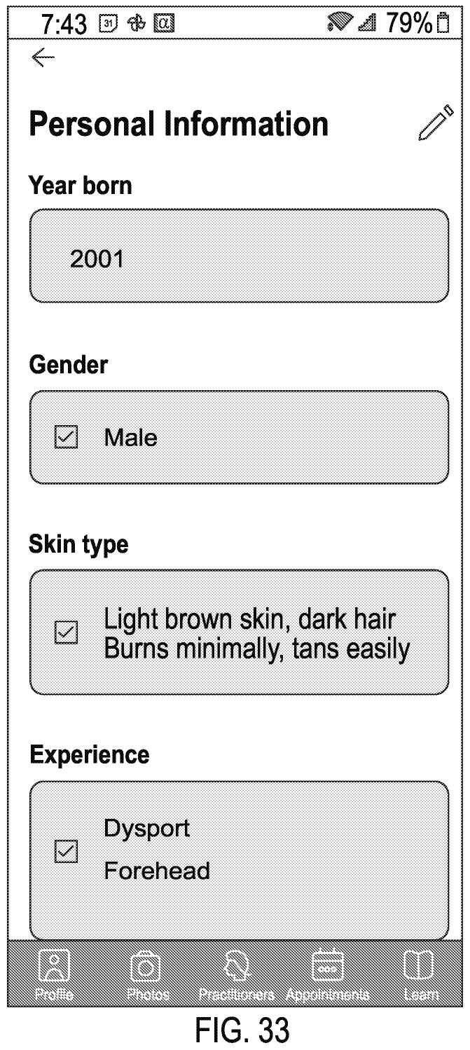

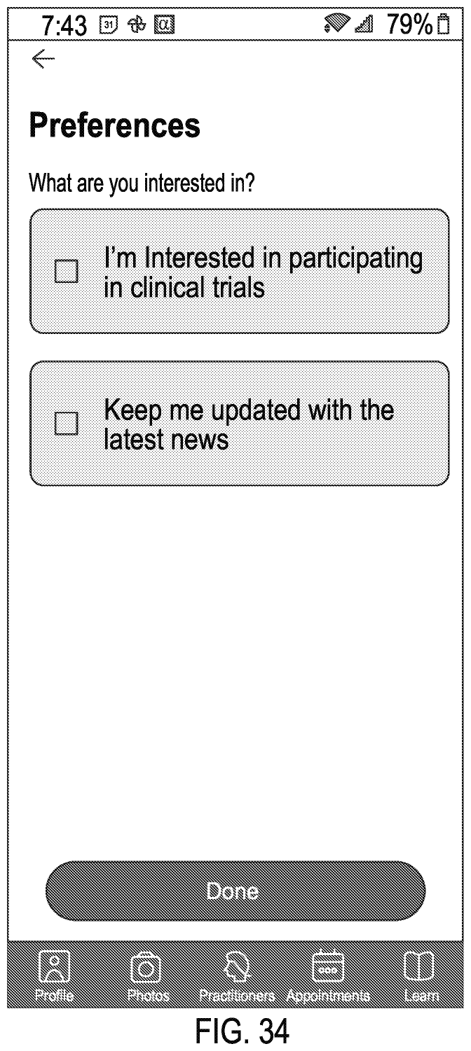

In some examples, a system of the present disclosure may be configured to utilize various combinations of one or more of the above classifications and/or information during the training and/or prediction steps for various users of the system. For example, when a new patient creates a profile (e.g., using the example mobile application of any of FIGS. 20-36, etc.), an example system of the present disclosure may be configured to execute an algorithm that uses various combinations of the information and/or classifications described above to create a predictive model for that specific new patient. For instance, based on a user's profile (e.g., skin type, gender, age, severity grade, etc.), the example system (e.g., via the mobile application, remote server, etc.) may identify individuals with similar profiles that used X units of Y product to achieve a Grade Z in W days, and required another treatment after A weeks or months to maintain that Grade Z. The example system may then provide similar recommendations to the new user based on the identification of these other individuals.

In some implementations, the example system may be configured to capture a current image of a new user and use images associated with similar profiles in the database (FIG. 8) to create a predictive image model for that new user (e.g., simulated image, etc.) that simulates potential improvements that the user may experience by using a certain recommended treatment.

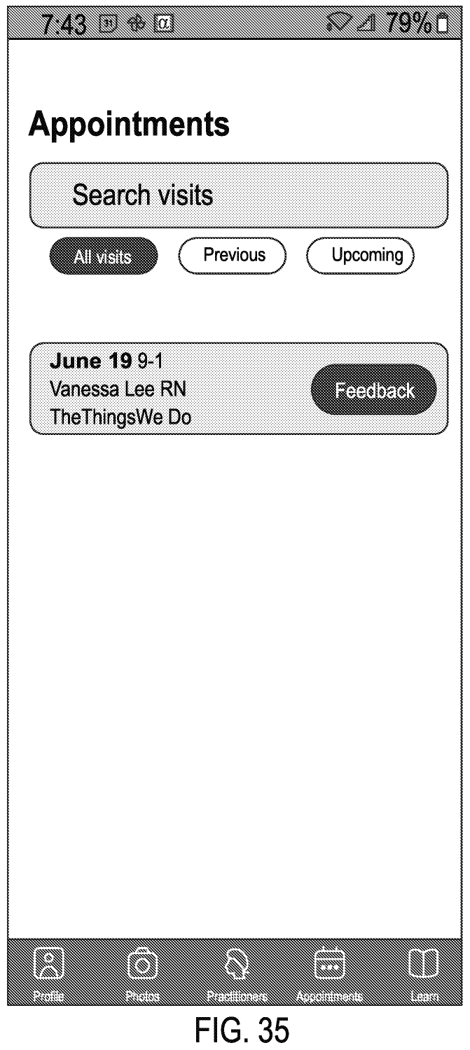

FIGS. 20A, 20B, 2C, 21, 22A, 22B, 22C, 22D, 23, 24A, 24B, 25, 26, 27A, 27B, 28A, 28B, 29, 30, 31, 32, 33, 34, 35, and 36 illustrate another example graphical user interface of an example mobile application of an example system, in accordance with the present disclosure. An example method of the present disclosure is as follows. In this example, a user first opens the example mobile application (FIGS. 20A-20C) of the example system of the present disclosure. In this example, the user is instructed how the app works: Teaching the user about facial and body aesthetics, assessing the user's wrinkles on face and neck (and/or issues with other parts of the user's body) in real time by the example AI/Machine Learning based engine of the example system, providing specific recommendations to the user based on the assessment, offering to connect the user with a practitioner within certain proximity of the user, and managing appointments (FIG. 35) and follow-up with the user through the example mobile application.

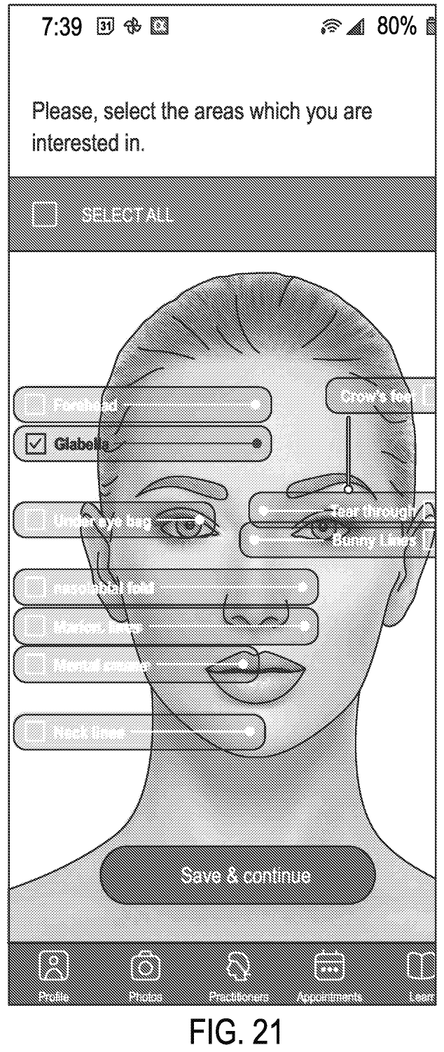

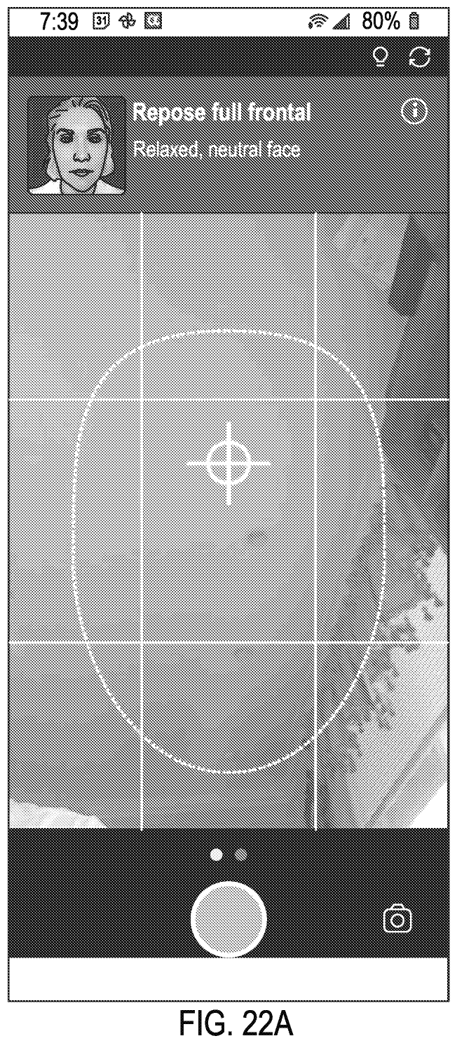

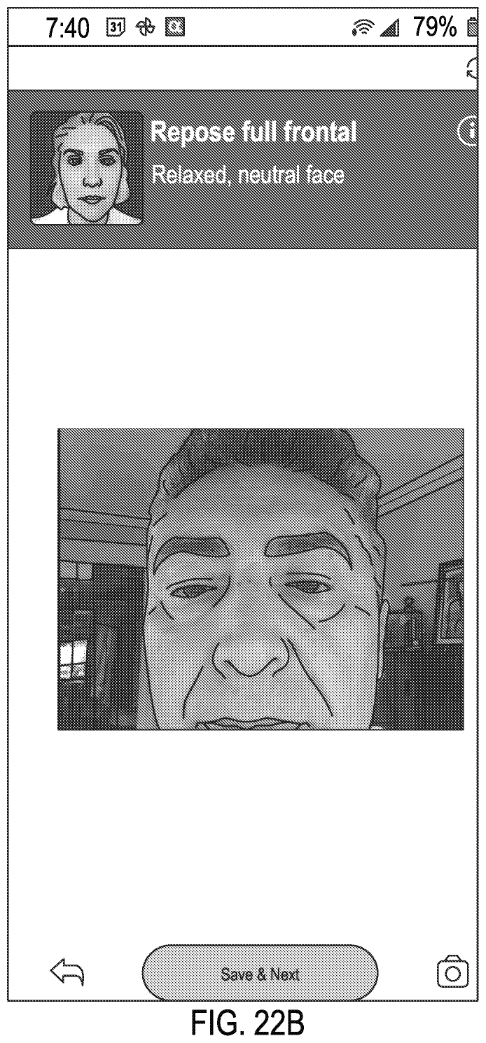

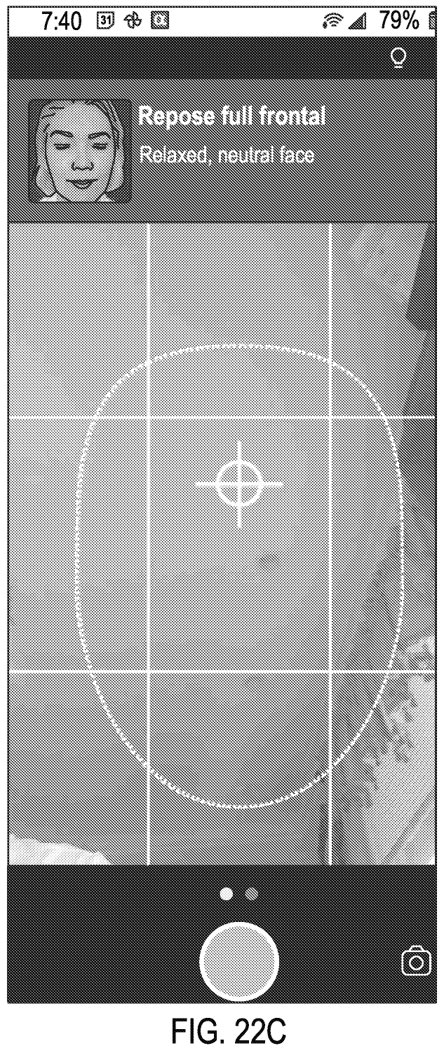

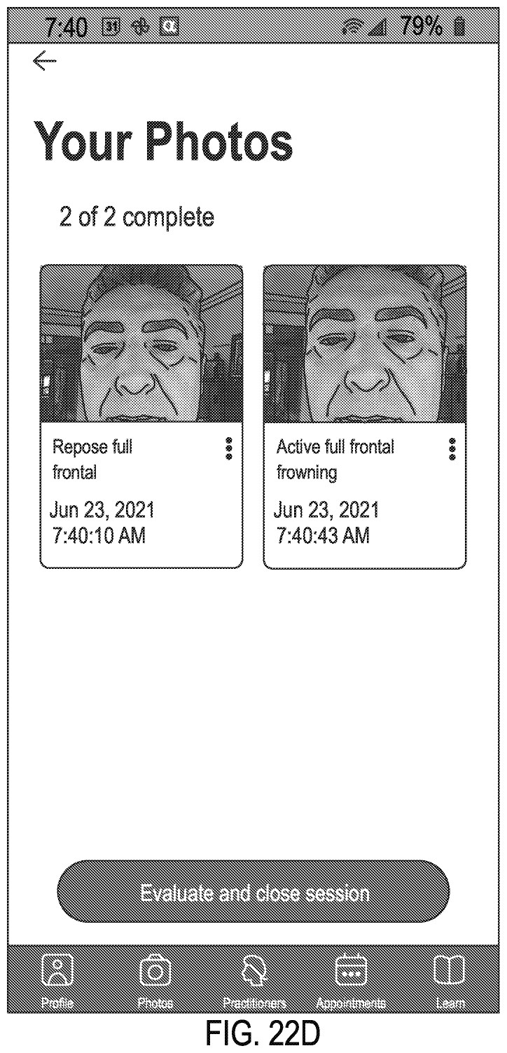

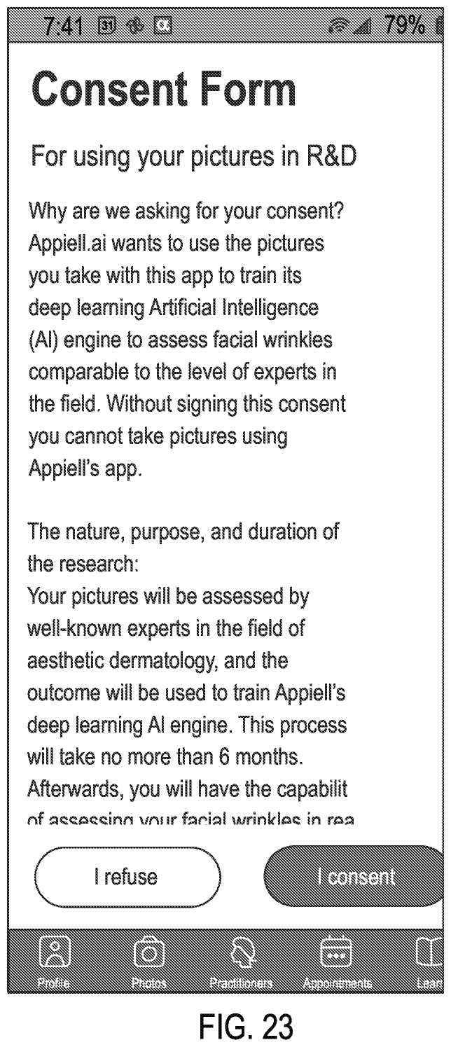

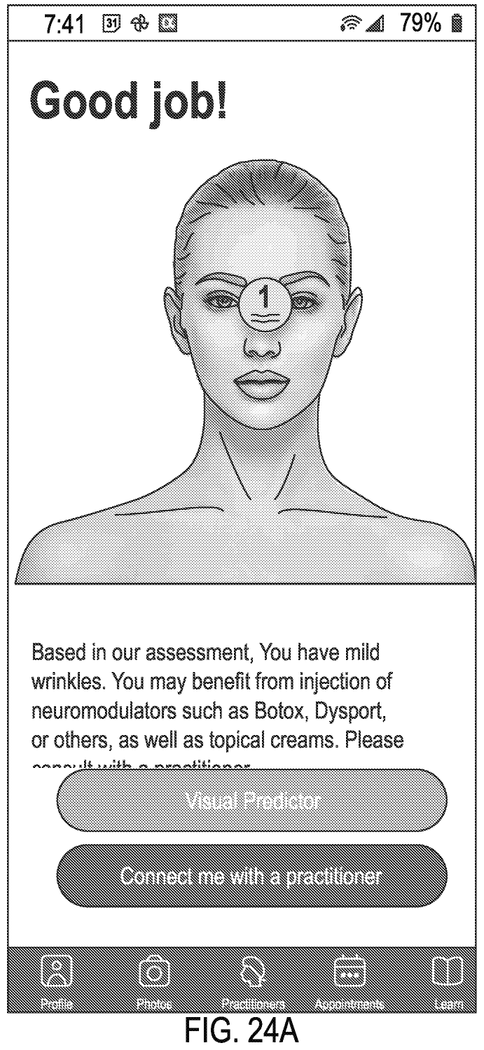

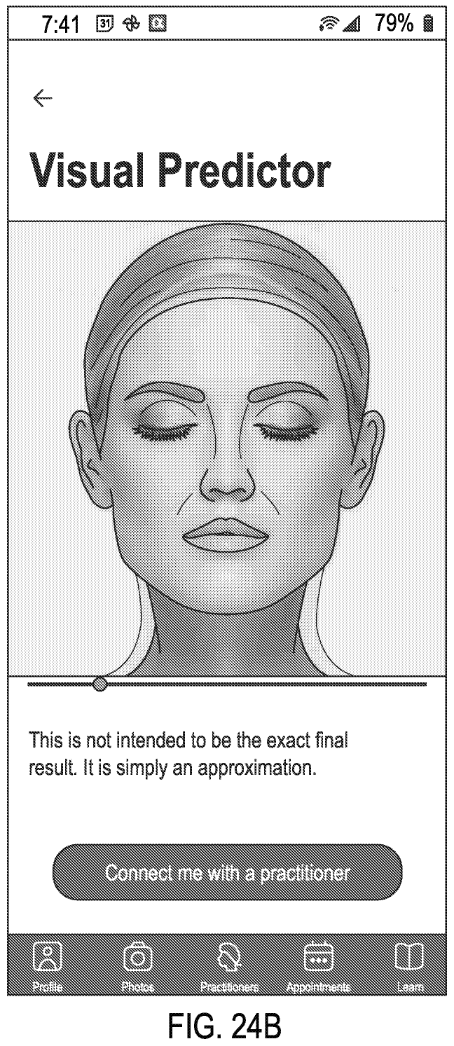

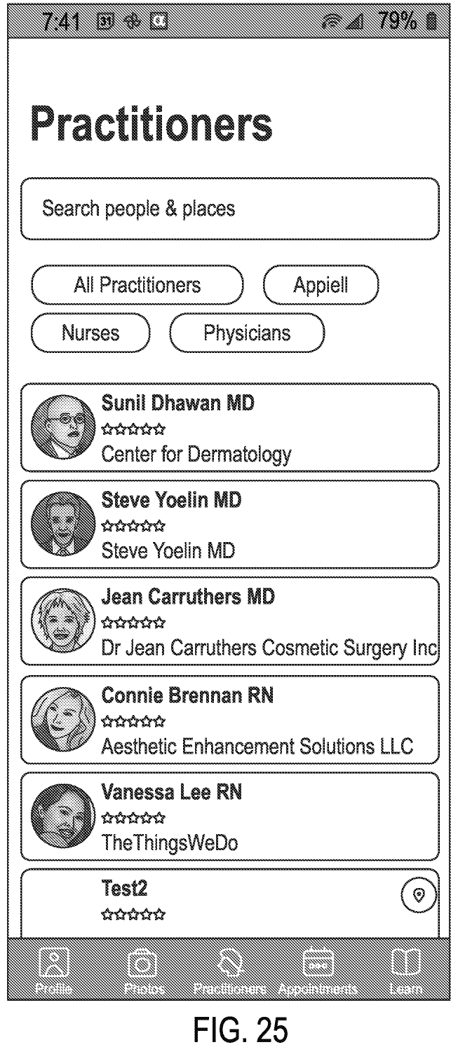

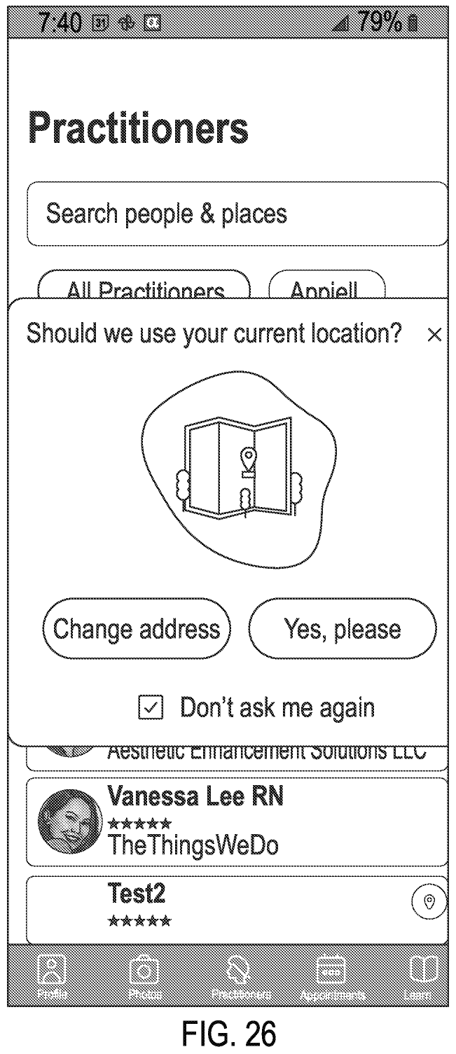

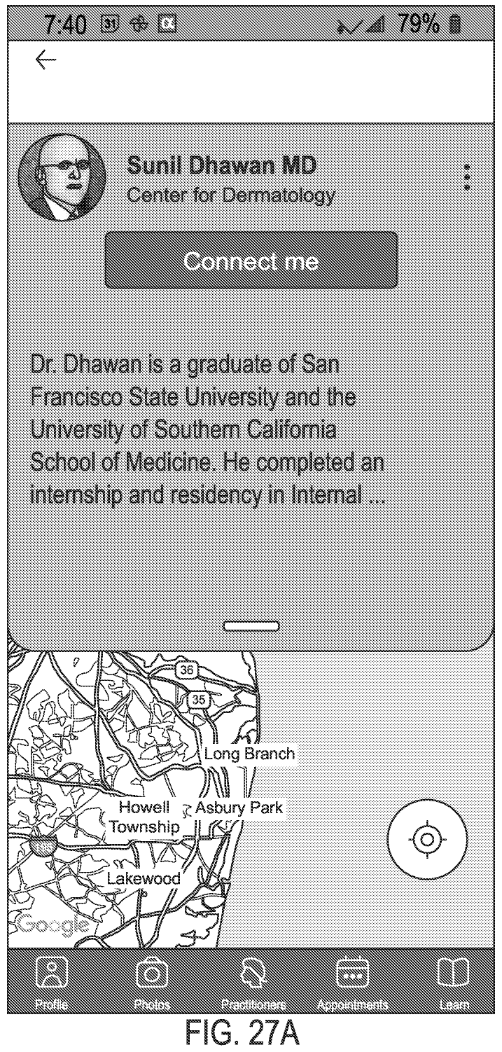

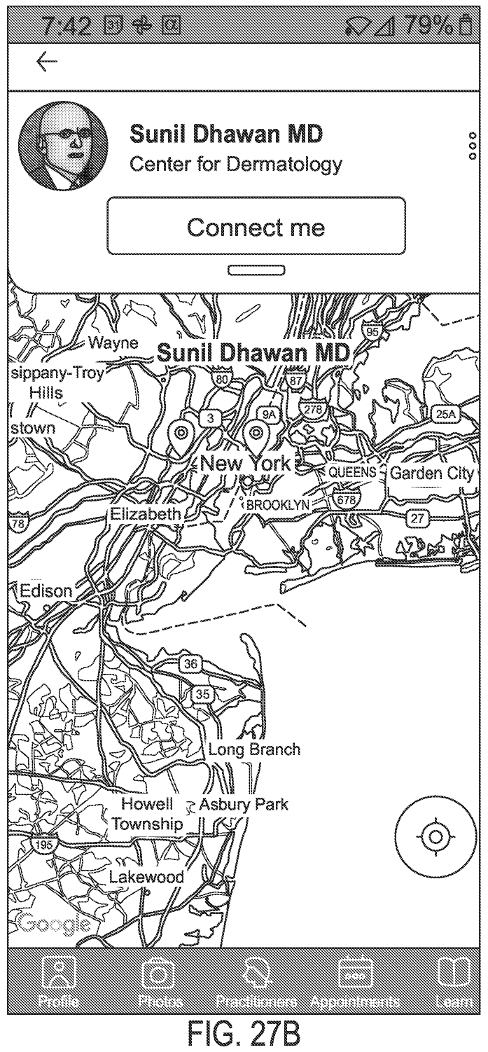

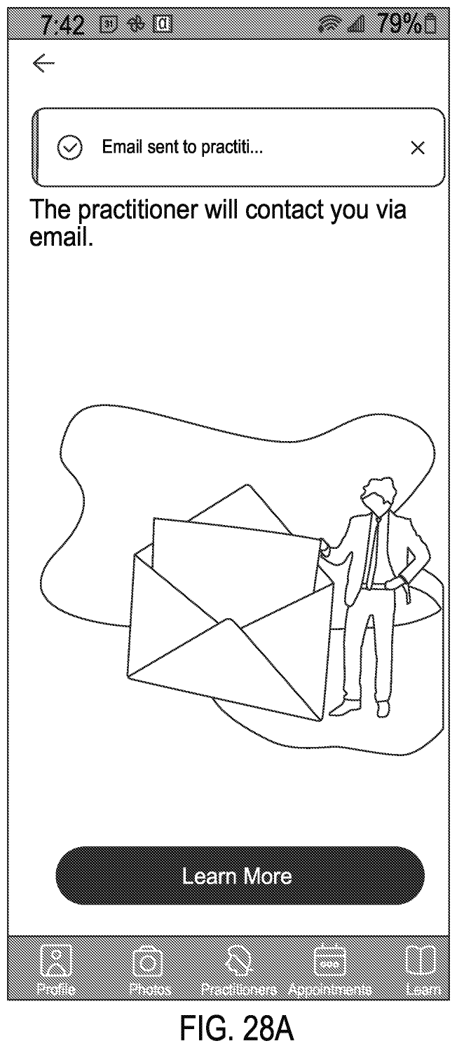

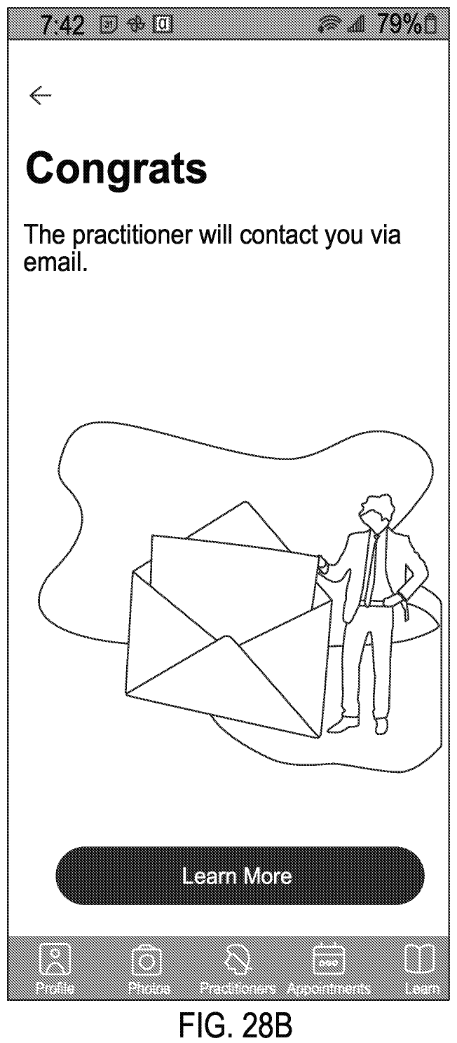

Continuing with the example method, via the example mobile application, the user selects (FIG. 21) the areas of her face and/or body the user is interested in. Based on the user's selections, the example system presents (FIGS. 22A-22D), to the user via the example mobile application, several structured poses that she needs to take pictures of her face/body (either selfies or assisted with a friend when selfies are not possible/adequate). In this example, the pictures are then uploaded (FIG. 23) by the example mobile application to the AI/Deep learning engine of the example system. In this example, the AI/Deep learning engine then assesses the pictures and returns back individual scores for each area selected (e.g., Glabella, Forehead, Crow's Feet, etc.), which may then be presented to the user, along with specific treatment recommendations (FIG. 24A-24B) and options for the user specific case, via the example mobile application. In some examples, the example method also determines and provides (FIG. 25) a set of practitioner suggestions to the user. The set of practitioners may be suggested (FIG. 26) to the user, for example, based on either the user's present geo-location (extracted from the user's mobile device) or a specific address the user types in through the example mobile application. In some examples, the user may then select (FIGS. 27A-27B) a practitioner from the provided set of practitioners presented in the example mobile application. In some examples, the example method may involve enabling the user to specify a proximity (e.g., 5, 10, 20, . . . miles radius). In some examples, practitioners can also be filtered by the example system based on professional degree (e.g., Physician or Nurse, provider network, etc.) and/or any other filtering criteria presented to the user via an interface of the example mobile application, in accordance with the present disclosure. Once the user selects the practitioner, in some examples, the example method involves managing communications (FIGS. 28A-28B) with the selected practitioner's office, setting appointments, and/or reminding the user to keep the appointment (FIG. 35), etc.

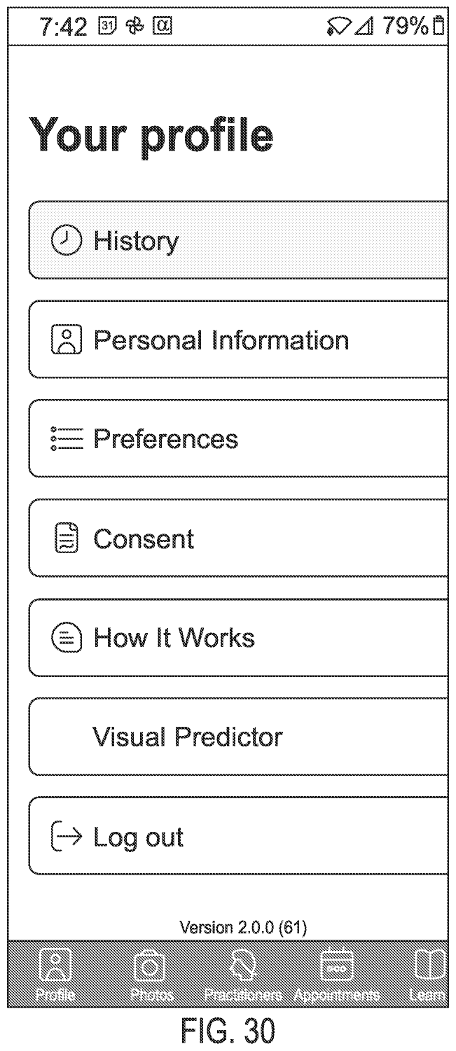

In some examples, for first time users, in addition to obtaining the facial/body structured pictures, the example method involves enabling the user to complete the user's profile (e.g., personal information such as Birth year, gender, skin type based on the Fitzpatrick types, etc.) and providing a questionnaire to the user indicating various preferences and interests, such as interest in participation in clinical trials (FIGS. 30-34).

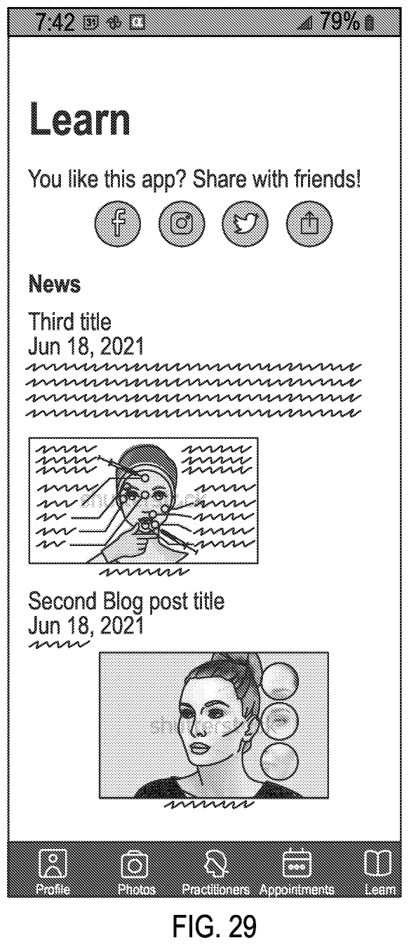

In some examples, for a returning user, the example method provides (e.g., via the example mobile application) to the user the picture taking and assessment features, connecting with a practitioner features, and/or learning features (FIG. 29) of the example system. In some examples, the example system enables the user to alter and/or update his/her profile through the Profile tab in the example mobile application (FIGS. 30-34).

In some examples, the example mobile application includes a LEARN tab (FIG. 29). Under the learn tab, the example system may be configured to present to the user a curated list of blogs, articles, videos, generational information about Aesthetics, and/or other learning material. This information may be gathered, collated, and curated by the example system from various sources such as sources across the internet, as well as articles produced by one or more affiliated experts.

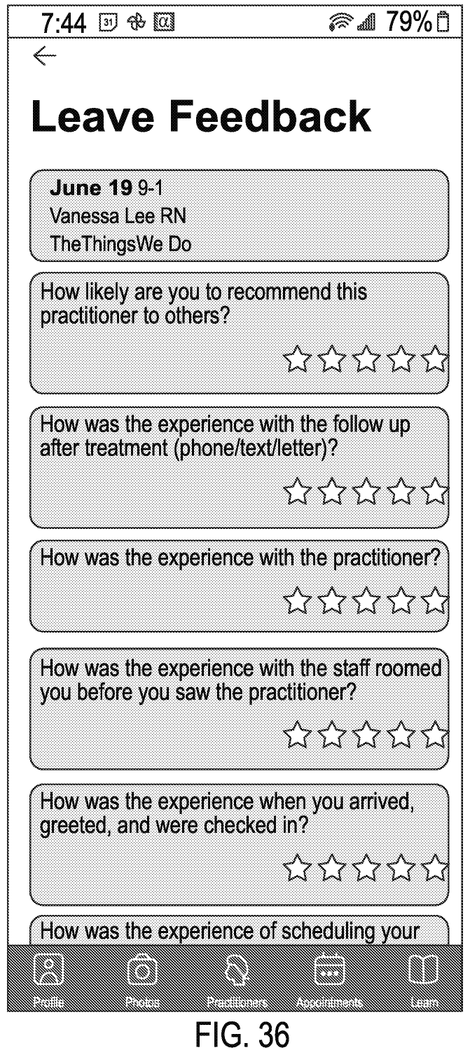

Practitioner feedback (FIG. 36): In some examples, the example system enables the user to rate a practitioner across several dimensions. In some examples, the example system may share this feedback information with one or more other users of the example mobile application.

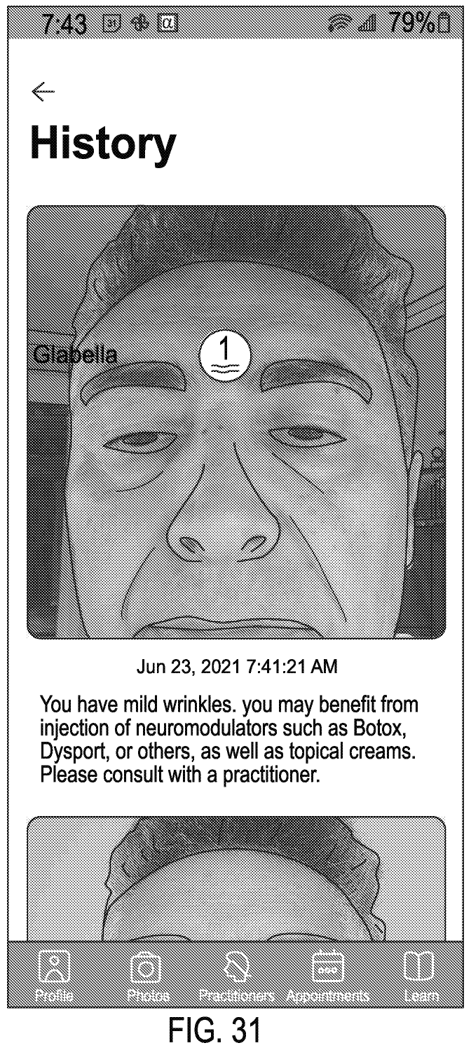

HISTORY (FIG. 31): In some examples, the example system (e.g., under a HISTORY tab of the example mobile application) enables the user to access all (or some) of the user's past pictures and assessments taken through the example mobile application of the example system. This allows the user (and optionally other users of the data such as clinical drug development companies using this data, etc.) to see the impact of any drug/cream/injection/filler used on a continuous time basis on the user. Currently the user is only seen once every several months by the practitioner, so no facility other than frequent visits to the practitioner's office exists today to provide such frequent assessment of the progression of (e.g., improvement or worsening of) the user's condition(s).

Structured database of users: In some examples, the example system deposits all information related to the user in one or more databases (FIG. 8). In some examples, this information can then be retrieved by interested parties such as drug development companies to examine in great detail information about the usage of the drugs. For example, certain ethnicities with certain skin types may need more doses of an injection to achieve the same result as some other skin types. Thus, the example system can be used to perform analysis on user data to identify various types of correlations and/or other type of information pertaining to skin conditions.

FACIAL SCORE: In some examples, based on example mathematical computations disclosed herein, the example system may generate an overall facial and/or body score for a user based on various individual areas (e.g., facial, body, etc.) assessed by the example system for the user. This is akin to something like a FICO score in finance, for instance, and can then be used for a variety of aesthetics applications and beyond.

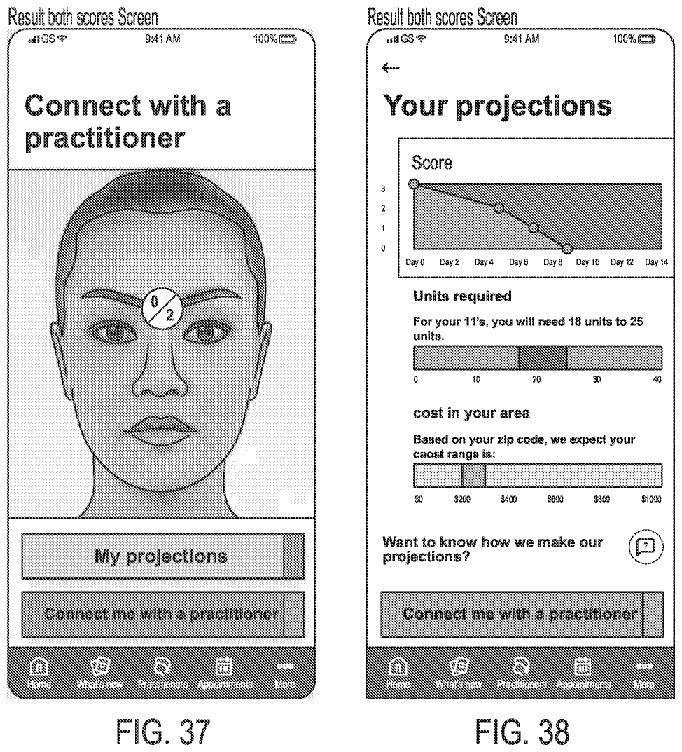

FIGS. 37 and 38 illustrate yet another example graphical user interface of an example mobile application of an example system, in accordance with the present disclosure. In this example, the system of the present method may present a result screen (FIG. 37), similarly to FIG. 24A, which allows the user to select a practitioner in line with the describe above. Additionally, the results screen of FIG. 37 may have an option for showing a projections screen (FIG. 38) that provides predictive information to the user if the user follows a recommended treatment. For example, as shown the projections screen of FIG. 38, an example system of the present disclosure may be configured to predict, based on the user's age bracket, gender, and/or skin type, etc., how many (and/or a range of) units (e.g., dose, etc.) of a certain product that the user should use to improve a grade of her wrinkles (e.g., from grade 3 to grade 0) and the amount of time (e.g., chart showing progress from day 0 to day 5 in FIG. 38) until that progress is achieved. The example mobile application may also be configured to display the range or quantity of units (e.g., injections (e.g., see middle range bar in FIG. 38) that patients with similar demographics have used in the past to achieve such results as well as a prediction of the costs associated with the recommended treatment (e.g., bottom range bar in FIG. 38) based on a zip code of the user.

In some examples, although not shown, the example mobile application may additionally or alternatively show a chart (e.g., similar to the top chart in FIG. 38) that indicates both repose (relaxed) scores and active (activating muscles causing wrinkles) scores predicted for the user in the projections screen of FIG. 38.

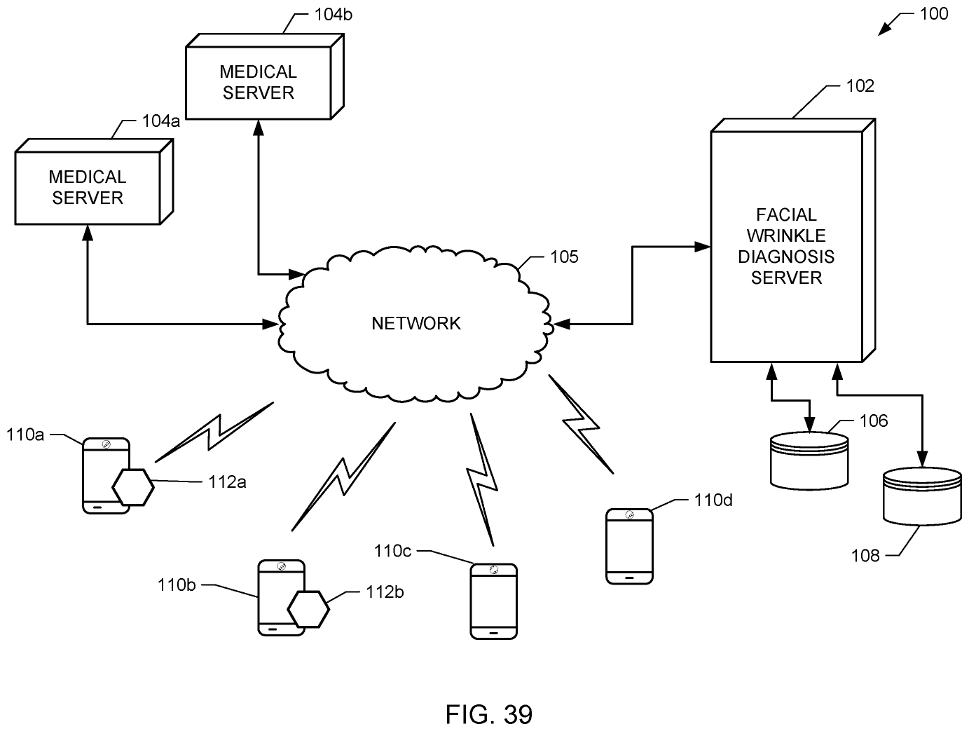

FIG. 39 shows a diagram of a facial wrinkle diagnosis system 100, according to an example embodiment of the present disclosure. In the illustrated example, the facial wrinkle diagnosis system 100 is implemented as a computing system that includes a facial wrinkle diagnosis server 102 communicatively coupled (via a network 105) to one or more medical servers 104a, 104b, and one or more computing devices 110a, 110b, 110c, 110d (collectively referred to herein as user devices 110). The system 100 also includes one or more memory devices 106, 108.

The server 102 and the user devices 110 may include one or more processors, such as single core processors, multi-core processors, etc., and a memory (e.g., memory devices 106, 108, etc.) storing instructions that are executable by the one or more processors to perform the functions described in the present disclosure.

In some examples, the server 102 receives, accesses, and/or compiles facial wrinkle image data (e.g., images of facial wrinkle regions, etc.) associated with a plurality of persons, including users associated with the user devices 110 and/or a different population of people (e.g., anonymized subjects of a research study, etc.). The server 102 may also receive measurements (specific to a user or specific to a group of one or more images collected in a session) of the facial wrinkle characteristics of the user (e.g., values selected from the scales in FIGS. 12-19, etc.). By way of example, a measurement of a particular type of facial wrinkles (e.g., globular lines, etc.) for a particular user can be entered by an expert for training a machine learning model or neural network model of the system 100. In some examples, the server 102 may receive one or more measurements (e.g., facial wrinkle diagnosis, etc.) from the medical server 104a and/or 104b (e.g., medical reports from a dermatologist, etc.).

In some examples, the server 102 includes one or more machine learning algorithms and/or analytic processors for processing the image data (obtained from the user devices 110) to evaluate facial wrinkle characteristics (e.g., a score from the scales shown in FIGS. 12-19, etc.), and/or other information (e.g., description of the severity of the facial wrinkles such as mild, moderate, etc.) tailored for a particular user. In some examples, the one or more machine learning algorithms executing on the server 102 may be configured to use the expert-entered facial wrinkle diagnosis data and/or the previously collected user-specific image data to develop a data model that characterizes a relationship between image features and facial wrinkle conditions. In some examples, the server 102 may also generate a recommended treatment to improve the skin conditions of a particular user (e.g., based on treatments used by other similar users in the past); and may also optionally generate a computer-modified image predicting an appearance of the user if he or she follows the recommended treatment (e.g. see the computer-generated image of FIG. 24B).

As noted above, the example server 102 may be communicatively coupled to one or more servers 104 and configured to receive at least some user data. The medical servers 104 may be associated with one or more medical systems, which transmit user information (e.g., dermatologist reports, prescription history, etc.) and/or other health information related to a user of the system 100. The user-specific information may be transmitted over the network 105, such as the Internet, a cellular network, other network, or a combination of one or more networks.

The memory devices 106, 108 can be used to store user data (e.g., characterizing relationships between images or image features and facial wrinkle characteristics of a population of people), the images themselves, other user data, and/or program instructions executable by one or more processors of the server 102 (and/or the user devices 110) to perform the functions described above. To that end, the memory devices 106 and 108 may include any computer-readable medium, such as random access memory (“RAM”), read only memory (“ROM”), flash memory, magnetic or optical disks, optical memory, or other storage media.

In some examples, one or more functions described above as being performed by the health management server 102 may alternatively or additionally be performed by one or more of the user devices 110.

In some examples, one or more of the user devices 110a-d may include input/output devices 110a, 110b, etc., such as cameras (for capturing images of users), displays (for displaying the various GUIs of the previous Figures), etc.

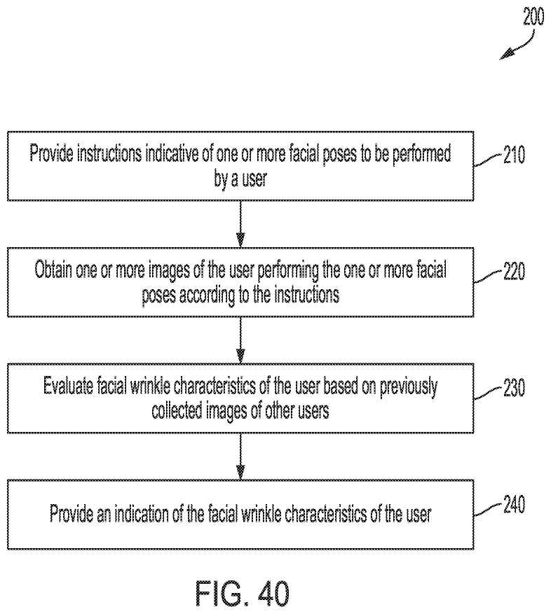

FIG. 40 illustrates a flowchart of an example method 200 for facial wrinkle diagnosis, according to an example embodiment of the present disclosure. Although the example method 200 is described with reference to the flowchart illustrated in FIG. 40, it will be appreciated that many other methods of performing the acts associated with the method 200 may be used. For example, the order of some of the blocks may be changed, certain blocks may be combined with other blocks, one or more blocks may be repeated, and/or some of the blocks may be removed. The method 200 may be performed by processing logic that may comprise hardware (circuitry, dedicated logic, etc.), software, or a combination of both. For example, method 300 can be implemented using the facial wrinkle diagnosis server 102, the user devices 110, and/or one or more other components illustrated in FIG. 39.

At block 210, the method 200 involves providing instructions indicative of one or more facial poses to be performed by a user. For example, a user device 110 may present instructions (e.g., FIG. 4A) to the user about a requested view or viewing angle, as well as one or more facial poses (e.g., frown, rest, constricted muscle, etc.) (e.g., FIGS. 4B-4C).

At block 220, method 200 involves obtaining one or more images of the user performing the one or more facial poses according to the instructions (see e.g., FIGS. 5A-5C).

At block 230, method 200 involves evaluating facial wrinkle characteristics of the user based on previously collected images of other users. For example, in line with the discussion above, the system 100 and/or the system of FIG. 8 can train a machine learning data model (e.g., neural network data model) with images of a facial region from various users (e.g., the images shown in FIGS. 12-16, etc.) tagged with a classification (e.g., an expert-assigned score from 0-3 such as those shown in FIGS. 12-18). The system 100 (e.g., the server 102) can then use the image data and the classification data to analyze the images (e.g., using any type of image processing/feature extraction technique) and thus develop a data model that characterizes a relationship between certain features in the images that result in a certain value from the 0-4 scales shown in FIGS. 12-18.

Further, the system 100 may similarly train multiple data models for each different facial region. For example, the method 200 may involve mapping a range of coordinates (e.g., any of the regions bounded with squares in FIG. 11) to a portion of an image captured by a user and identify the mapped portion as corresponding to that particular region (e.g., the glabellar region shown in FIG. 12 is mapped only to portions of the images taken by the users corresponding to that particular region).

At block 240, the method 200 involve providing an indication of the facial wrinkle characteristics of the user. As shown in FIG. 24A for example, the system 100 may evaluate the facial wrinkle characteristics of the user by providing a written description (e.g., mild wrinkles, etc.) to the user. Alternatively or additionally, the system 100 performing the method 200 may also determine and provide an indication of a recommended treatment (e.g., botox, etc.). The system of method 200 may also optionally generate a computer-generated modified image of the user (e.g., FIG. 24B) that predicts the appearance of the user if the user follows the recommended treatment.

In some examples, a system of method 200 may also provide additional information (e.g., see FIGS. 37-38), such as recommended practitioners near a location of the user, expected costs, and/or projection time charts for when the user's skin condition (e.g., facial wrinkle score between 0-3) improve if the user adopts the proposed treatment (e.g., see FIG. 38).

### Example Methods of Treatment

### Definitions

“Administration,” or “to administer” means the step of giving (i.e. administering) a pharmaceutical composition or active ingredient to a subject. The pharmaceutical compositions disclosed herein can be administered via a number of appropriate routs, including oral and intramuscular or subcutaneous routes of administration, such as by injection, topically, or use of an implant.

“Botulinum toxin” or “botulinum neurotoxin” or “BoNT” means a neurotoxin derived from Clostridium botulinum, as well as modified, recombinant, hybrid and chimeric botulinum toxins. A recombinant botulinum toxin can have the light chain and/or the heavy chain thereof made recombinantly by a non-Clostridial species. “Botulinum toxin,” as used herein, encompasses the botulinum toxin serotypes A (“BoNT/A”), B (“BoNT/B”), C (“BoNT/C”), D (“BoNT/D”), E (“BoNT/E”), F (“BoNT/F”), G (“BoNT/G”), and H (“BoNT/H”). “Botulinum toxin,” as used herein, also encompasses both a botulinum toxin complex (i.e. the 300, 600 and 900 kDa complexes) as well as pure botulinum toxin (i.e. the about 150 kDa neurotoxic molecule), all of which are useful in the practice of the disclosed embodiments.

“Clostridial neurotoxin” means a neurotoxin produced from, or native to, a Clostridial bacterium, such as Clostridium botulinum, Clostridium butyricum or Clostridium beratti, as well as a Clostridial neurotoxin made recombinantly by a non-Clostridial species.

“Dermal filler” or “injectable filler” as used herein means a soft tissue filler injected into the skin at different depths to help fill in, for example, wrinkles, provide volume, and augment features. Most of these fillers are temporary because they are eventually absorbed by the body. Most dermal fillers today consist of hyaluronic acid, a naturally occurring polysaccharide that is present in skin and cartilage. Fillers are typically made of sugar molecules or composed of hyaluronic acids, collagens (which may come from pigs, cows, cadavers, or may be generated in a laboratory), the person's own transplanted fat, and biosynthetic polymers. Examples of the latter include calcium hydroxylapatite, polycaprolactone, polymethylmethacrylate, and polylactic acid.

“Fast-acting neurotoxin” as used herein refers to a botulinum toxin that produces effects in the patient more rapidly than those produced by, for example, a botulinum neurotoxin type A. For example, the effects of a fast-acting botulinum toxin (such as botulinum type E) can be produced within 36 hours.

“Fast-recovery neurotoxin” as used herein refers to a botulinum toxin that whose effects diminish in the patient more rapidly than those produced by, for example, a botulinum neurotoxin type A. For example, the effects of a fast-recovery botulinum toxin (such as botulinum type E) can diminish within, for example, 120 hours, 150 hours, 300 hours, 350 hours, 400 hours, 500 hours, 600 hours, 700 hours, 800 hours, or the like. It is known that botulinum toxin type A can have an efficacy for up to 12 months, and in some circumstances for as long as 27 months, when used to treat glands, such as in the treatment of hyperhidrosis. However, the usual duration of an intramuscular injection of a botulinum neurotoxin type A is typically about 3 to 4 months.

“Neurotoxin” means a biologically active molecule with a specific affinity for a neuronal cell surface receptor. Neurotoxin includes Clostridial toxins both as pure toxin and as complexed with one or more non-toxin, toxin-associated proteins.

“Patient” means a human or non-human subject receiving medical or veterinary care.

“Pharmaceutical composition” means a formulation in which an active ingredient can be, for example, a neurotoxin such as a Clostridial toxin, an injectable filler, or combinations thereof. The word “formulation” means that there is at least one additional ingredient (such as, for example and not limited to, an albumin [such as a human serum albumin (HSA) or a recombinant human albumin] and/or sodium chloride) in the pharmaceutical composition in addition to a Clostridial (for example, a botulinum neurotoxin) active ingredient. A pharmaceutical composition is therefore a formulation which is suitable for diagnostic, therapeutic or cosmetic administration to a subject, such as a human patient. The pharmaceutical composition can be in a lyophilized or vacuum dried condition, a solution formed after reconstitution of the lyophilized or vacuum dried pharmaceutical composition with saline or water, for example, or as a solution that does not require reconstitution. As stated, a pharmaceutical composition can be liquid, semi-solid, or solid. A pharmaceutical composition can be animal-protein free.

“Purified botulinum toxin” means a pure botulinum toxin or a botulinum toxin complex that is isolated, or substantially isolated, from other proteins and impurities which can accompany the botulinum toxin as it is obtained from a culture or fermentation process. Thus, a purified botulinum toxin can have at least 95%, and more preferably at least 99% of the non-botulinum toxin proteins and impurities removed.

“Therapeutic formulation” means a formulation that can be used to treat and thereby alleviate a disorder or a disease and/or symptom associated thereof.

“Therapeutically effective amount” means the level, amount or concentration of an agent (e.g. such as a Clostridial toxin or pharmaceutical composition comprising clostridial toxin) needed to treat a disease, disorder or condition without causing significant negative or adverse side effects.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived symptom, disease, disorder or condition.

“Unit” or “U” means an amount of active botulinum neurotoxin standardized to have equivalent neuromuscular blocking effect as a Unit of commercially available botulinum neurotoxin type A (for example, Onabotulinumtoxin A (BOTOX©)).

### Methods of Treatment

Disclosed embodiments comprise use of an artificial intelligence (AI) platform for the diagnosis of a condition, disorder, symptom, or disease, in a subject. Further embodiments comprise evaluation of the severity of a condition, disorder, symptom, or disease, in a subject. For example, in disclosed embodiments, facial wrinkles such as glabellar lines or horizontal frown lines can be diagnosed. Similarly, in disclosed embodiments, the severity of facial wrinkles such as glabellar lines or horizontal frown lines can be evaluated.

In embodiments, methods disclosed herein can further comprise methods for treating the symptom, condition, disease or disorder diagnosed using an AI platform. In embodiments, treating the symptom, condition, disease, or disorder can comprise alleviation, prevention, or reduction of a symptom, condition, disease, or disorder.

For example, embodiments disclosed herein can comprise reduction of local muscular activity and thereby reduction of the appearance of cosmetic imperfections or irregularities, for example facial lines. In embodiments the cosmetic irregularities can comprise glabellar lines, horizontal frown lines, forehead lines, “bunny” lines, smile irregularities, chin irregularities, platysmal bands, “marionette” lines, lip lines, crows feet, eyebrow irregularities, combinations thereof, and the like. For example, disclosed embodiments comprise diagnosis and treatment of a skin symptom, condition, disease, or disorder, such as wrinkles, for example facial wrinkles, such as glabellar lines.

Administration sites useful for practicing the disclosed embodiments can comprise the glabellar complex, including the corrugator supercilli and the procerus; the obicularis oculi; the superolateral fibers of the obicularis oculi; the frontalis; the nasalis; the levator labii superioris aleque nasi; the obicularis oris; the masseter; the depressor anguli oris; and the platysma.

Disclosed embodiments can comprise treatment of, for example, skin disorders, for example, acne, and the like.

Disclosed embodiments can comprise treatment of inflammatory skin diseases. For example, disclosed embodiments can comprise treatment of rosacea, psoriasis, eczema, and the like, following diagnosis or evaluation using an AI platform.

Disclosed embodiments can promote the production of, for example, elastin, collagen, and the like. Disclosed embodiments can comprise methods of increasing the elasticity of the skin following diagnosis or evaluation using an AI platform.

Disclosed embodiments can comprise administration of a dermal filler. For example, in embodiments, treatment of lower-than-desired lip volume can comprise administration of a dermal filler, for example hyaluronic acid following diagnosis or evaluation using an AI platform.

Disclosed embodiments can comprise a surgical procedure. For example, in embodiments, an appropriate surgical procedure can be performed following diagnosis or evaluation using an AI platform.

Disclosed embodiments can comprise treatment of a hair loss disorder. For example, in embodiments, an appropriate treatment can be performed following diagnosis or evaluation using an AI platform.

### Neurotoxin Compositions

Embodiments disclosed herein comprise neurotoxin compositions. Such neurotoxins can be formulated in any pharmaceutically acceptable formulation in any pharmaceutically acceptable form. The neurotoxin can also be used in any pharmaceutically acceptable form supplied by any manufacturer. Disclosed embodiments comprise use of Clostridial neurotoxins.

The Clostridial neurotoxin can be made by a Clostridial bacterium, such as by a Clostridium botulinum, Clostridium butyricum, or Clostridium beratti bacterium. Additionally, the neurotoxin can be a modified neurotoxin; that is a neurotoxin that has at least one of its amino acids deleted, modified or replaced, as compared to the native or wild type neurotoxin. Furthermore, the neurotoxin can be a recombinantly produced neurotoxin or a derivative or fragment thereof.

In disclosed embodiments, the neurotoxin is formulated in unit dosage form; for example, it can be provided as a sterile solution in a vial or as a vial or sachet containing, for example, a lyophilized powder for reconstituting in a suitable vehicle, such as saline for injection.

In embodiments the neurotoxin, for example botulinum toxin, is formulated in a solution containing saline and pasteurized HSA, which stabilizes the toxin and minimizes loss through non-specific adsorption. The solution can be sterile filtered (0.2 μm filter), filled into individual vials, and then vacuum-dried to give a sterile lyophilized powder. In use, the powder can be reconstituted by the addition of sterile unpreserved normal saline (sodium chloride 0.9% for injection).

In an embodiment, botulinum type A is supplied in a sterile solution for injection with a 5-mL vial nominal concentration of 20 ng/mL in 0.03 M sodium phosphate, 0.12 M sodium chloride, and 1 mg/mL HSA, at pH 6.0.

Although the composition may only contain a single type of neurotoxin, for example botulinum type A, disclosed compositions can include two or more types of neurotoxins, which can provide enhanced therapeutic effects in treating the disorders. For example, a composition administered to a patient can include botulinum types A and E, or A and B, or the like. Administering a single composition containing two different neurotoxins can permit the effective concentration of each of the neurotoxins to be lower than if a single neurotoxin is administered to the patient while still achieving the desired therapeutic effects. This type of “combination” composition can also provide benefits of both neurotoxins, for example, quicker effect combined with longer duration.

The composition administered to the patient can also contain other pharmaceutically active ingredients, such as, protein receptor or ion channel modulators, in combination with the neurotoxin or neurotoxins. These modulators may contribute to the reduction in neurotransmission between the various neurons. For example, a composition may contain gamma aminobutyric acid (GABA) type A receptor modulators that enhance the inhibitory effects mediated by the GABA_{A} receptor. The GABA_{A} receptor inhibits neuronal activity by effectively shunting current flow across the cell membrane. GABA_{A} receptor modulators may enhance the inhibitory effects of the GABA_{A} receptor and reduce electrical or chemical signal transmission from the neurons. Examples of GABA_{A} receptor modulators include benzodiazepines, such as diazepam, oxaxepam, lorazepam, prazepam, alprazolam, halazeapam, chordiazepoxide, and chlorazepate. Compositions may also contain glutamate receptor modulators that decrease the excitatory effects mediated by glutamate receptors. Examples of glutamate receptor modulators include agents that inhibit current flux through AMPA, NMDA, and/or kainate types of glutamate receptors. Further disclosed compositions comprise esketamine.

Disclosed neurotoxin compositions can be injected into the patient using a needle or a needleless device. In certain embodiments, the method comprises sub-dermally injecting the composition in the individual. For example, administering may comprise injecting the composition through a needle of, in embodiments, no greater than about 30 gauge. In embodiments, the injection should be made in a perpendicular manner using a 23 to 27 gauge sclerotherapy or similar needle with a tip length of, for example, 2-5 mm. In certain embodiments, the method comprises administering a composition comprising a botulinum toxin, for example botulinum toxin type A.

Administration of the disclosed compositions can be carried out by syringes, catheters, needles and other means for injecting. The injection can be performed on any area of the mammal's body that is in need of treatment, however disclosed embodiments contemplate injection into the patient's stomach and the vicinity thereof. The injection can be into any specific area such as epidermis, dermis, fat, smooth or skeletal muscle, nerve junction, or subcutaneous layer.

More than one injection and/or sites of injection may be necessary to achieve the desired result. Also, some injections, depending on the location to be injected, may require the use of fine, hollow, Teflon®-coated needles. In certain embodiments, guided injection is employed, for example by electromyography, or ultrasound, or fluoroscopic guidance or the like.

The frequency and the amount of toxin injection under the disclosed methods can be determined based on the nature and location of the particular area being treated. In certain cases, however, repeated injection may be desired to achieve optimal results. The frequency and the amount of the injection for each particular case can be determined by the person of ordinary skill in the art.

Although examples of routes of administration and dosages are provided, the appropriate route of administration and dosage are generally determined on a case by case basis by the attending physician. Such determinations are routine to one of ordinary skill in the art (see for example, Harrison's Principles of Internal Medicine (1998), edited by Anthony Fauci et al., 14th edition, published by McGraw Hill). For example, the route and dosage for administration of a Clostridial neurotoxin according to the present disclosed invention can be selected based upon criteria such as the solubility characteristics of the neurotoxin chosen as well as the intensity and scope of the condition being treated.

### Injectable Fillers

Disclosed embodiments can comprise use of injectable fillers. Such fillers can be formulated in any pharmaceutically acceptable formulation in any pharmaceutically acceptable form. The injectable filler can also be used in any pharmaceutically acceptable form supplied by any manufacturer.

### Training Methods

Disclosed embodiments comprise methods of training a practitioner to identify or gauge a symptom, condition, disease or disorder. For example, in embodiments, a practitioner is trained in the evaluation of facial wrinkles by grading the severity of the wrinkles as indicated, for example, in a photograph. Disclosed training methods comprise comparing the practitioner's evaluation with an AI-produced evaluation.

### Data Quality Control

Disclosed embodiments comprise methods performing quality control on data, for example data associated with a clinical trial. For example, in embodiments, collected clinical trial data can be evaluated using an AI platform to identify irregularities.

### Neurotoxin Dosages

The neurotoxin can be administered in an amount of between about 10-3 U/kg and about 35 U/kg. In an embodiment, the neurotoxin is administered in an amount of between about 10⁻² U/kg and about 25 U/kg. In another embodiment, the neurotoxin is administered in an amount of between about 10-1 U/kg and about 15 U/kg. In another embodiment, the neurotoxin is administered in an amount of between about 1 U/kg and about 10 U/kg. In many instances, an administration of from about 1 unit to about 300 Units of a neurotoxin, such as a botulinum type A, provides effective therapeutic relief. In an embodiment, from about 50 Units to about 400 Units of a neurotoxin, such as a botulinum type A, can be used and in another embodiment, from about 100 Units to about 300 Units of a neurotoxin, such as a botulinum type A, can be locally administered into a target tissue.

In embodiments, administration can comprise a total dose per treatment session of about 100 Units of a botulinum neurotoxin, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or about 320 Units, or about 340 Units, or about 360 Units, or about 380 Units, or about 400 Units, or about 450 Units, or about 500 Units, or the like.

In embodiments, administration can comprise a total dose per treatment session of not less than 100 Units of a botulinum neurotoxin, or not less than 110 Units, or not less than 120 Units, or not less than 130 Units, or not less than 140 Units, or not less than 150 Units, or not less than 160 Units, or not less than 170 Units, or not less than 180 Units, or not less than 190 Units, or not less than 200 Units, or not less than 210 Units, or not less than 220 Units, or not less than 230 Units, or not less than 240 Units, or not less than 250 Units, or not less than 260 Units, or not less than 270 Units, or not less than 280 Units, or not less than 290 Units, or not less than 300 Units, or not less than 320 Units, or not less than 340 Units, or not less than 360 Units, or not less than 380 Units, or not less than 400 Units, or not less than 450 Units, or not less than 500 Units, or the like.

In embodiments, administration can comprise a total dose per treatment session of not more than 100 Units of a botulinum neurotoxin, or not more than 110 Units, or not more than 120 Units, or not more than 130 Units, or not more than 140 Units, or not more than 150 Units, or not more than 160 Units, or not more than 170 Units, or not more than 180 Units, or not more than 190 Units, or not more than 200 Units, or not more than 210 Units, or not more than 220 Units, or not more than 230 Units, or not more than 240 Units, or not more than 250 Units, or not more than 260 Units, or not more than 270 Units, or not more than 280 Units, or not more than 290 Units, or not more than 300 Units, or not more than 320 Units, or not more than 340 Units, or not more than 360 Units, or not more than 380 Units, or not more than 400 Units, or not more than 450 Units, or not more than 500 Units, or the like.

In embodiments, the total dose administered to the target sites can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, the total dose administered to the target sites can be, for example, about 30 Units of a botulinum neurotoxin, or about 40 Units, or about 50 Units, or about 60 Units, or about 70 Units, or about 80 Units, or about 90 Units, or about 100 Units, or about 110 Units, or about 120 Units, or about 130 Units, or about 140 Units, or about 150 Units, or about 160 Units, or about 170 Units, or about 180 Units, or about 190 Units, or about 200 Units, or about 210 Units, or about 220 Units, or about 230 Units, or about 240 Units, or about 250 Units, or about 260 Units, or about 270 Units, or about 280 Units, or about 290 Units, or about 300 Units, or the like.

In embodiments, the total dose administered to the target sites can be, for example, at least 30 Units of a botulinum neurotoxin, at least 40 Units, at least 50 Units, at least 60 Units, at least 70 Units, at least 80 Units, at least 90 Units, at least 100 Units, at least 110 Units, at least 120 Units, at least 130 Units, at least 140 Units, at least 150 Units, at least 160 Units, at least 170 Units, at least 180 Units, at least 190 Units, at least 200 Units, at least 210 Units, at least 220 Units, at least 230 Units, at least 240 Units, at least 250 Units, at least 260 Units, at least 270 Units, at least 280 Units, at least 290 Units, at least 300 Units, or the like.

In embodiments, the total dose administered to the target sites can be, for example, not more than 30 Units of a botulinum neurotoxin, not more than 40 Units, not more than 50 Units, not more than 60 Units, not more than 70 Units, not more than 80 Units, not more than 90 Units, not more than 100 Units, not more than 110 Units, not more than 120 Units, not more than 130 Units, not more than 140 Units, not more than 150 Units, not more than 160 Units, not more than 170 Units, not more than 180 Units, not more than 190 Units, not more than 200 Units, not more than 210 Units, not more than 220 Units, not more than 230 Units, not more than 240 Units, not more than 250 Units, not more than 260 Units, not more than 270 Units, not more than 280 Units, not more than 290 Units, not more than 300 Units, or the like.

In embodiments, administration can comprise a total dose per year of not more than 800 Units of a neurotoxin, for example botulinum type A neurotoxin, or not more than 900 Units, or not more than 1000 Units, or not more than 1200 Units, or not more than 1400 Units, or the like.

In embodiments, the dose of the neurotoxin is expressed in protein amount or concentration. For example, in embodiments the neurotoxin can be administered in an amount of between about 0.2 ng and 20 ng. In an embodiment, the neurotoxin is administered in an amount of between about 0.3 ng and 19 ng, about 0.4 ng and 18 ng, about 0.5 ng and 17 ng, about 0.6 ng and 16 ng, about 0.7 ng and 15 ng, about 0.8 ng and 14 ng, about 0.9 ng and 13 ng, about 1.0 ng and 12 ng, about 1.5 ng and 11 ng, about 2 ng and 10 ng, about 5 ng and 7 ng, and the like, into a target tissue such as a muscle.

Ultimately, however, both the quantity of toxin administered and the frequency of its administration will be at the discretion of the physician responsible for the treatment and will be commensurate with questions of safety and the effects produced by the toxin.

Disclosed embodiments comprise treatments that can be repeated. For example, a repeat treatment can be performed when the patient begins to experience symptoms of gastroparesis. However, preferred embodiments comprise repeating the treatment prior to the return of symptoms. Therefore, disclosed embodiments comprise repeating the treatment, for example, after 6 weeks, 8 weeks, 10 weeks, 12 weeks, 14 weeks, 16 weeks, 18 weeks, 20 weeks, 22 weeks, 24 weeks, or more. Repeat treatments can comprise administration sites that differ from the administration sites used in a prior treatment.

A controlled release system can be used in the embodiments described herein to deliver a neurotoxin in vivo at a predetermined rate over a specific time period. A controlled release system can be comprised of a neurotoxin incorporated into a carrier. The carrier can be a polymer or a bio-ceramic material. The controlled release system can be injected, inserted or implanted into a selected location of a patient's body and reside therein for a prolonged period during which the neurotoxin is released by the implant in a manner and at a concentration which provides a desired therapeutic efficacy.

Polymeric materials can release neurotoxins due to diffusion, chemical reaction or solvent activation, as well as upon influence by magnetic, ultrasound or temperature change factors. Diffusion can be from a reservoir or matrix. Chemical control can be due to polymer degradation or cleavage of the drug from the polymer. Solvent activation can involve swelling of the polymer or an osmotic effect.

A kit for practicing disclosed embodiments is also encompassed by the present disclosure. The kit can comprise a 30 gauge or smaller needle and a corresponding syringe. The kit can also comprise a Clostridial neurotoxin composition, such as a botulinum type A toxin composition. The neurotoxin composition may be provided in the syringe. The composition is injectable through the needle. The kits are designed in various forms based the sizes of the syringe and the needles and the volume of the injectable composition(s) contained therein, which in turn are based on the specific deficiencies the kits are designed to treat.

### EXAMPLES

The following non-limiting Examples are provided for illustrative purposes only in order to facilitate a more complete understanding of representative embodiments. This example should not be construed to limit any of the embodiments described in the present Specification.

### Example 1

### Glabellar Line Treatment

A 57 year old man is diagnosed with glabellar wrinkles using a disclosed AI platform. The man then undergoes treatment for glabellar lines with BoNT/A delivered at 5 injection sites in equal volumes (5 Units, 0.1 mL per site into the procerus, left and right medial corrugators, and left and right lateral corrugators). The appearance of the glabellar lines is reduced for 6 months.

### Example 2

### Glabellar Line Treatment

A 27 year old man is diagnosed with glabellar wrinkles using a disclosed AI platform. He then undergoes treatment for glabellar lines with BoNT/E delivered at 5 injection sites in equal volumes (5 Units, 0.1 mL per site into the procerus, left and right medial corrugators, and left and right lateral corrugators). The appearance of the glabellar lines is reduced for 7 months.

### Example 3

### Glabellar Line Treatment

A 47 year old woman is diagnosed with glabellar wrinkles using a disclosed AI platform. She then undergoes treatment with BoNT/B delivered at 5 injection sites in equal volumes (5 Units, 0.1 mL per site into the procerus, left and right medial corrugators, and left and right lateral corrugators). The appearance of the glabellar lines is reduced for 5 months.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

What is claimed is:

1. A method for facial wrinkle diagnosis, the method comprising:
providing, at a user device, instructions indicative of one or more facial poses to be performed by a user;
obtaining, via a camera, one or more images of the user performing the one or more facial poses according to the instructions;
based on previously collected images of other users performing the one or more facial poses and further based on previously stored user data indicative of facial wrinkle characteristics of the other users, evaluating facial wrinkle characteristics of the user; and
providing, at the user device, an indication of the facial wrinkle characteristics of the user.

2. The method of claim 1, further comprising:
training a machine learning data model to associate image features in the images of the other users with a user-input value in a scale indicative of a severity of facial wrinkles indicated in each respective image; and
providing image data associated with the one or more images as input to the machine learning data model to generate an output vale in the scale indicative of the facial wrinkle characteristics of the user predicted by the machine learning model.

3. The method of claim 1, further comprising:
identifying a first image portion in the one or more images corresponding to a first facial region associated with a first type of facial wrinkles; and
evaluating facial wrinkle characteristics of the user with respect to the first facial region relative to facial wrinkle characteristics in the first facial region within the other images of the other users.

4. The method of claim 3, wherein identifying the first image portion comprises mapping the one or more images to a range of coordinates assigned to the first facial region in a face mesh model.

5. The method of claim 4, further comprising:
identifying a second image portion in the one or more images corresponding to a second facial region associated with a second type of facial wrinkles; and
evaluating facial wrinkle characteristics of the user with respect to the second facial region relative to facial wrinkle characteristics in the second facial region within the other images of the other users/

6. The method of claim 3, wherein the first type of facial wrinkles comprises glabellar frown lines.

7. The method of claim 3, wherein the first type of facial wrinkles comprises crows feet lines.

8. The method of claim 3, wherein the first type of facial wrinkles comprises crows feet lines.

9. The method of claim 3, wherein the first type of facial wrinkles comprises forehead lines.

10. The method of claim 3, wherein the first type of facial wrinkles comprises Under eye bag lines.

11. The method of claim 3, wherein the first type of facial wrinkles comprises nasolabial fold lines.

12. The method of claim 3, wherein the first type of facial wrinkles comprises marionette lines.

13. The method of claim 3, wherein the first type of facial wrinkles comprises lip mental crease lines.

14. The method of claim 3, wherein the first type of facial wrinkles comprises bunny lines.

15. The method of claim 3, wherein the first type of facial wrinkles comprises tear trough lines.

16. The method of claim 3, wherein the first type of facial wrinkles comprises neck lines.

17. The method of claim 1, further comprising:
generating a score indicative of a relative condition of facial wrinkles in the face of the user; and
providing the score for display at the user device.
